(19) United States
(12) Patent Application Publication
(10) Pub. No.: US 2026/0083944 A1
(43) Pub. Date: Mar. 26, 2026

(54) INFLATION CONTROL DEVICES FOR A BALLOON CATHETER, SUCH AS FOR USE IN CLOT TREATMENT SYSTEMS

(71) Applicant: Inari Medical, Inc., Irvine, CA (US)

(72) Inventors: Benjamin Merritt, San Clemente, CA (US); Jared Shimizu, Tustin, CA (US)

(21) Appl. No.: 19/334,650

(22) Filed: Sep. 19, 2025

Related U.S. Application Data
(60) Provisional application No. 63/697,007, filed on Sep. 20, 2024.

(51) Int. Cl. A61M 25/10; A61B 17/22; A61M 39/22

(52) U.S. Cl. CPC A61M 25/10185; A61B 17/22; A61M 39/22; A61B 2017/22067; A61M 2025/1052; A61M 2205/3334; A61M 2210/12

(57) ABSTRACT

Disclosed herein are clot treatment systems including mechanical thrombectomy devices and clot treatment devices, and associated devices and methods. In some embodiments, a method of removing clot material from a blood vessel of a patient includes inserting a catheter to proximate the clot material within the blood vessel while radially constraining a clot treatment device and an embolic protection device within the catheter. The clot treatment device can include a balloon catheter including an inflation port positioned on a proximal end of the balloon catheter and fluidly connected to a balloon on a distal end thereof. The balloon catheter includes an inflation control device fluidly connected to the inflation port, wherein the inflation control device is configured to reduce pressure within the balloon during inflation of the balloon.

### CROSS-REFERENCE TO RELATED APPLICATION

This application claims the benefit of U.S. Provisional Patent Application No. 63/697,007, filed Sep. 20, 2024, and titled “INFLATION CONTROL DEVICE FOR A BALLOON CATHETER,” which is incorporated herein by reference in its entirety.

### TECHNICAL FIELD

The present technology generally relates to balloon catheters, and more particularly to balloon catheters with inflation control devices and associated systems and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

In the arterial circulation (e.g., the peripheral arterial circulation, the pulmonary circulation), the undesirable material can cause harm by obstructing different arteries. For example, an obstruction within the pulmonary arteries is a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

Other arterial thromboembolic medical conditions include acute limb ischemia (ALI), acute visceral ischemia, and chronic limb ischemia (CLI). ALI is characterized a sudden decrease in blood flow to a limb caused by a blood clot (e.g., thromboembolism) obstructing the arteries supplying blood to the limb. If not treated properly, ALI can lead to tissue damage, organ dysfunction, and/or limb loss. Acute visceral ischemia is characterized by a sudden decrease in blood flow to the organs in the abdominal cavity (e.g., visceral organs) caused by a blood clot obstructing the arteries supplying blood to the organs. If not treated properly, acute visceral ischemia can lead to tissue damage, organ dysfunction, and/or other damage to a patient's arterial system. CLI is a long-term reduction in blood flow to the limbs caused by a blood clot in the arteries supplying blood to the arms and/or legs. If not treated properly, CLI can lead to pain, numbness, weakness, and/or impaired wound healing in the arms and legs.

Treatment of arterial thromboembolic medical conditions often requires open surgical procedures and/or the use of lytic therapy. Such procedures can result in distal embolization of the blot material, vessel trauma, and significant blood loss. Mechanical thrombectomy catheters can also be used to treat arterial thromboembolic medical conditions, and often include an element on the distal end which serves to capture the thromboembolism. The element may be made from wire, laser cut metal including nitinol, looped or braided wire, or an inflated element such as a balloon. However, the element on the distal end of the mechanical thrombectomy catheter can cause embolization of the clot material as the element is inserted and/or retracted through the clot material. The embolized clot material can flow to other parts of the body, which may lead to other medical complications.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a side view of a clot treatment system configured in accordance with embodiments of the present technology.

FIG. 2A is a perspective view of a clot treatment device of the clot treatment system of FIG. 1 in accordance with embodiments of the present technology.

FIGS. 2B and 2C are side and top views, respectively, of the clot treatment device of FIG. 2A in accordance with embodiments of the present technology.

FIG. 2D is a plan view of the clot treatment device of FIG. 2A in a pre-shaped configuration in accordance with embodiments of the present technology.

FIG. 2E is an enlarged side view of a leading edge portion of the clot treatment device of FIG. 2A in accordance with embodiments of the present technology.

FIG. 2F is another plan view of the clot treatment device of FIG. 2A in the pre-shaped configuration in accordance with embodiments of the present technology.

FIG. 3A is a side view of a coupling portion of the clot treatment device of FIG. 2A in accordance with embodiments of the present technology.

FIG. 3B is a plan view of the coupling portion of the clot treatment device of FIG. 3A in a pre-shaped configuration in accordance with embodiments of the present technology.

FIGS. 4 and 5 are side views of coupling portions of a clot treatment device in pre-shaped configurations in accordance with additional embodiments of the present technology.

FIG. 6A is a plan view of a clot treatment device configured in accordance with additional embodiments of the present technology.

FIG. 6B is an enlarged view of a portion of the clot treatment device of FIG. 6A in accordance with embodiments of the present technology.

FIG. 7 is a perspective view of a relief feature of a clot treatment device in accordance with embodiments of the present technology.

FIG. 8 is a top view of a clot treatment device in accordance with additional embodiments of the present technology.

FIG. 9 is a top view of a clot treatment device in accordance with additional embodiments of the present technology.

FIG. 10 is a flow diagram of a process or method for removing clot material from the vasculature of a patient using a clot treatment system in accordance with embodiments of the present technology.

FIGS. 11A-11G are side views of a clot treatment system during different stages of the process or method of FIG. 10 in accordance with embodiments of the present technology.

FIGS. 12-16 illustrate respective clot treatment cleaning tools in accordance with embodiments of the present technology.

FIG. 17 is a flow diagram of a process or method for removing clot material from a clot treatment device using a cleaning tool in accordance with embodiments of the present technology.

FIGS. 18 and 19 are side views of a proximal portion of an embolic protection device configured in accordance with embodiments of the present technology.

FIG. 20 is a perspective view of an embolic protection device configured in accordance with additional embodiments of the present technology.

FIG. 21 is a side view of an embolic protection device configured in accordance with additional embodiments of the present technology.

FIG. 22 is a perspective view of an embolic protection device configured in accordance with additional embodiments of the present technology.

FIG. 23 is a perspective view of an embolic protection device configured in accordance with additional embodiments of the present technology.

FIG. 24 is a perspective view of a balloon catheter assembly configured in accordance with embodiments of the present technology.

FIG. 25A is a side view of an inflation control device in accordance with embodiments of the present technology. FIGS. 25B and 25C are cross-sectional views of the inflation control device of FIG. 25A in a closed position and an open position, respectively, in accordance with embodiments of the present technology.

FIG. 26A is a side view of the inflation control device of FIG. 25A assembled with additional adapters in accordance with embodiments of the present technology.

FIG. 26B is a perspective view of the inflation control device of FIG. 25A fluidly connected between the syringe and the inflation port of the assembly of FIG. 24 in accordance with embodiments of the present technology.

FIG. 27A is a perspective view of the inflation control device of FIG. 25A mounted to the inflation port of the assembly of FIG. 24 in accordance with embodiments of the present technology.

FIG. 27B is a perspective view of the inflation control device of FIG. 27A with the syringe fluidly connected to the inlet of the inflation control device in accordance with embodiments of the present technology.

FIG. 28A is a side view of an inflation control device in a first position in accordance with additional embodiments of the present technology.

FIG. 28B is an enlarged cross-sectional view of the inflation control device in the first position of FIG. 28A in accordance with embodiments of the present technology.

FIG. 29A is a side view of the inflation control device of FIG. 28A in a second position in accordance with embodiments of the present technology.

FIG. 29B is an enlarged cross-sectional view of the inflation control device in the second position of FIG. 29A in accordance with embodiments of the present technology.

FIGS. 30A and 30B are front and back perspective views, respectively, of the restrictor of the inflation control device of FIG. 28A in accordance with embodiments of the present technology.

FIG. 30C is a perspective view of a restrictor of the inflation control device of FIG. 28A in accordance with additional embodiments of the present technology.

FIG. 31 is a perspective view of the assembly of FIG. 24 including two inflation control devices configured in accordance with embodiments of the present technology.

FIG. 32 is a side view of an inflation control device in accordance with additional embodiments of the present technology.

FIG. 33 is a first end view of the inflation control device of FIG. 32 in accordance with embodiments of the present technology.

FIG. 34 is a second end view of the inflation control device of FIG. 32 in accordance with embodiments of the present technology.

### DETAILED DESCRIPTION

### I. Introduction

The present technology is generally directed to clot treatment systems and associated devices and methods, including balloon catheter assemblies for embolic protection. In some embodiments, a balloon catheter assembly includes a balloon that can be inflated to restrict blood flow in a vessel via a pressure source. The pressure source can be fluidly connected to the balloon via an inflation control device configured to reduce the likelihood of over inflation of the balloon.

For example, an inflation control device in accordance with the present technology can include a housing including a flow path extending between an inlet fluidly connected to the pressure source and an outlet fluidly connected to the balloon. A restrictor can be positioned in the housing in the flow path between the inlet and the outlet, and the restrictor can be configured to move to a first position when fluid flows along the flow path from the inlet to the outlet and a second position when the fluid flows along the flow path from the outlet to the inlet. The restrictor can further define one or more first fluid channels and a second fluid channel. In the first position, the one or more first fluid channels are blocked such that the fluid flows from the inlet to the outlet (during inflation of the balloon) through only the second fluid channel. In the second position, the one or more first fluid channels are positioned such that the fluid flows from the outlet to the inlet (during deflation of the balloon) through the one or more first fluid channels and the second fluid channel. Accordingly, the inflation control device provides a greater flow rate during deflation of the balloon than during inflation of the balloon.

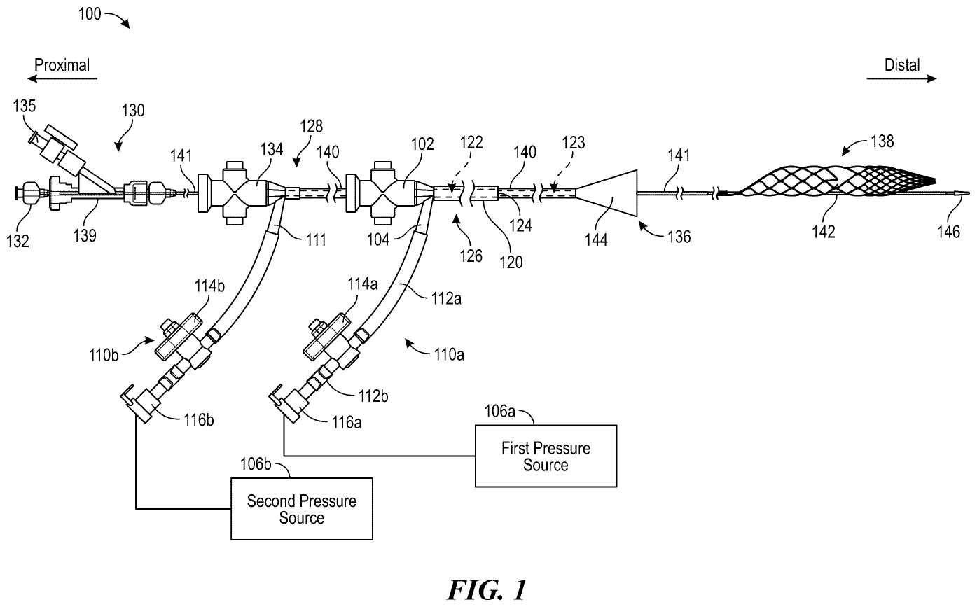

Certain details are set forth in the following description and in FIG. 1-34 to provide a thorough understanding of various embodiments of the present technology. In other instances, well-known structures, materials, operations, and/or systems often associated with intravascular procedures, clot removal procedures, clot treatment systems, clot treatment devices, embolic protection devices, catheters, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material (e.g., a thromboembolism), the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, first tubing assembly 110a is first introduced and discussed with reference to FIG. 1.

To the extent any materials incorporated herein by reference conflict with the present disclosure, the present disclosure controls.

### II. Selected Embodiments of Mechanical Thrombectomy Assemblies and Associated Methods

FIG. 1 is a partially schematic side view of a clot treatment system 100 (“the system 100”) configured in accordance with embodiments of the present technology. The system 100 can also be referred to as an aspiration assembly, a vascular access system, a clot removal system, a thrombectomy system, and/or the like. In some embodiments, the system 100 is configured for treating clot material (e.g., a thromboembolism) in a peripheral artery of a human patient, such as clot material leading to various medical conditions such as acute limb ischemia (ALI), acute visceral ischemia, chronic limb ischemia (CLI), and/or the like. In the illustrated embodiment, the system 100 includes an introducer catheter assembly 126, an embolic protection catheter assembly 128 (e.g., a funnel catheter assembly), and a mechanical thrombectomy catheter assembly 130. The system 100 (i) can include features generally similar in structure and/or function, or identical in structure and/or function, to those of the clot treatment systems described in detail in U.S. patent application Ser. No. 16/536,185, filed Aug. 8, 2019, and titled “SYSTEM FOR TREATING EMBOLISM AND ASSOCIATED DEVICES AND METHODS,” and U.S. patent application Ser. No. 18/349,674, filed Jul. 8, 2023, and titled “MECHANICAL THROMBECTOMY ASSEMBLY WITH EMBOLIC PROTECTION, AND ASSOCIATED DEVICES, SYSTEMS, AND METHODS,” each of which is incorporated herein by reference in its entirety, and/or (ii) can be used to treat/remove clot material from a patient (e.g., a human patient) using any of the methods described in detail therein.

In the illustrated embodiment, the introducer catheter assembly 126 includes a first catheter 120 defining a first lumen 122 (shown in dashed line in FIG. 1) extending entirely therethrough. The first catheter 120 is coupled to a first valve 102 such that the first valve 102 can be fluidly coupled to the first lumen 122, and first lumen 122 can extend from the first valve 102 to a distal terminus 124 of the first catheter 120. The first catheter 120 can be an introducer sheath configured to be inserted through the skin and tissue tract of the patient to provide an access site through which other components (e.g., the embolic protection catheter assembly 128, the mechanical thrombectomy catheter assembly 130) can traverse to easily access the vasculature. The embolic protection catheter assembly 128 can, similarly, include a second catheter 140 defining a second lumen 123 extending entirely therethrough and the second catheter 140 can also be coupled to a second valve 134, such that the second valve 134 can be fluidly coupled to the second lumen 123. The embolic protection catheter assembly 128 can further include an embolic protection device 136 coupled to the second catheter 140. The embolic protection device 136 illustrated in FIG. 1 includes a conical member or funnel 144.

The funnel 144 can include a conically-shaped polymer layer, a coil, one or more wires, a mesh, a braided structure, an at least partially coated and/or dipped structure, and/or another suitable structure. The funnel 144 can have a radial force range (normalized, e.g., per contact area in vessel diameter) of from about 3 Newtons to about 100 Newtons. When braided, the funnel 144 can have a braid angle of from about 75 degrees to about 150 degrees. The funnel 144 can comprise one or more polymers, such as polyurethane, silicone, and/or copolymers of polyurethane and silicone. All or a subset of the coils, wires, mesh, etc., used to form the funnel 144 can be radiopaque and/or can be coated with a radiopaque material, e.g., to improve imaging/visualization of the funnel 144 within the patient. For example, to give the funnel 144 radiopaque properties, a radiopaque substrate (e.g., barium sulfate, bismuth, tungsten, platinum, iridium, gold, and/or other radiopaque materials) can be added to a polyurethane salutation at a concentration of from about 5% to about 40% (by mass or volume) and then the funnel 144 can be dipped into the solution and cured. The radiopaque substrate could, additionally or alternatively, be electroplated onto the funnel 144 to form a radiopaque layer having a thickness of from about 10 μm to about 1000 μm. In these and/or other embodiment, all or a subset of the elements (e.g., wires) that form the funnel 144 can be radiopaque and/or one or more radiopaque elements (e.g., radiopaque wires) can be woven into the braided structure of the funnel 144. In these and/or other embodiments, the funnel 144 can be configured to inhibit or even prevent fluid (e.g., blood) flow (e.g., through one or more surfaces of the funnel 144) when deployed. That is, the funnel 144 can be impermeable to blood flow. For example, in some embodiments the funnel 144 includes a wire braid that is dip-coated to form an at least partially fluid-impermeable membrane around all, or at least a portion, of the wire braid. The coating can have a thickness of from about 50 μm to about 400 μm (for, e.g., braided funnels) and/or a thickness of from about 20 μm to about 400 μm (for, e.g., laser-cut or other unbraided funnels). All or at least a portion of the fluid that enters the funnel 144 (e.g., via passive flow, during aspiration, etc.) can be directed into the second lumen 123 of the second catheter 140. The funnel 144 can be self-expanding and configured to transition from a collapsed or undeployed configuration to an expanded or deployed configuration (shown in FIG. 1). For example, the funnel 144 can be in the collapsed configuration when positioned within a larger delivery catheter, such as the first catheter 120, and the funnel 144 can expand to the expanded configuration when the first catheter 120 (or other delivery catheter) is retracted over the second catheter 140 and/or the second catheter 140 is advanced so that the funnel 144 is no longer constrained by the first catheter 120 (when, e.g., the funnel 144 is positioned distally from the distal terminus 124 of the first catheter 120). In these and/or other embodiments, the embolic protection device 136 can include a balloon, a disc, a braided structure, and/or another suitable embolic protection device 136. The second valve 134 can be operably coupled to the embolic protection device 136 via the second catheter 140. Accordingly, moving the second valve 134, such as moving the second valve 134 in a proximal or distal direction, causes a corresponding movement of the embolic protection device 136.

In some embodiments, the first catheter 120 and/or the second catheter 140 (“catheters 120/140”) include an elongate member (e.g., a sheath, a shaft) configured to be inserted into and through a patient's vasculature and used to, for example, remove or otherwise treat clot material therein. The catheters 120/140, and/or one or more portions thereof, can have varying lengths, flexibilities, shapes, thicknesses, and/or other properties. For example, the catheters 120/140 can comprise one or more coils, braids, and/or other structures positioned between one or more liner layers (e.g., inner and outer liner layers). In some embodiments, the catheters 120/140 can include several features generally similar or identical in structure and/or function to any of the catheters described in U.S. patent application Ser. No. 17/529,018, titled “CATHETERS HAVING SHAPED DISTAL PORTIONS, AND ASSOCIATED SYSTEMS AND METHODS,” and filed Nov. 17, 2021, U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, and/or U.S. patent application Ser. No. 18/159,507, titled “ASPIRATION CATHETERS HAVING GROOVED INNER SURFACE, AND ASSOCIATE SYSTEM AND METHODS,” and filed Jan. 25, 2023, each of which is incorporated by reference herein in its entirety.

The first valve 102 and/or the second valve 134 (“valves 102/134”) can be integral with or coupled to the respective catheters 120/140 such that these components move together. In some embodiments, the valves 102/134 are hemostasis valves configured to maintain hemostasis during a clot treatment procedure by preventing fluid flow in a proximal direction through the valves 102/134 as various components such as dilators, delivery sheaths, pull members, guidewires, interventional devices, other aspiration catheters, and so on are inserted through the valves 102/134 to be delivered through the respective catheters 120/140 to a treatment site in a blood vessel. The first valve 102 can include a first branch or side port 104 configured to fluidly couple the first lumen 122 of the first catheter 120 to a first tubing assembly 110a. Similarly, the second valve 134 can include a second branch or side port 111 configured to fluidly couple the second lumen 123 of the second catheter 140 to a second tubing assembly 110b. In some embodiments, the valves 102/134 can be a valve of the type disclosed in U.S. Pat. No. 11,000,682, filed Aug. 30, 2018, and titled “HEMOSTASIS VALVES AND METHODS OF USE,” which is incorporated herein by reference in its entirety.

In the illustrated embodiment, a first tubing assembly 110a fluidly couples the first catheter 120 to a first pressure source 106a, such as a syringe. The first pressure source 106a can be configured to generate (e.g., form, create, charge, build-up) a vacuum (e.g., negative relative pressure) and store the vacuum for subsequent application to the first catheter 120. The first tubing assembly 110a can include one or more first tubing sections 112 (individually labeled as a first tubing section 112a and a second tubing section 112b), at least one first fluid control device 114a (e.g., a valve), and at least one first connector 116a (e.g., a Toomey tip connector) for fluidly coupling the first tubing assembly 110a to the first pressure source 106a and/or other suitable components. In some embodiments, the first fluid control device 114a is a stopcock that is fluidly coupled to (i) the first side port 104 of the first valve 102 via the first tubing section 112a and (ii) the first connector 116a via the second tubing section 112b. The first fluid control device 114 is externally operable by a user to regulate the flow of fluid therethrough and, specifically, from the first lumen 122 of the first catheter 120 to the first pressure source 106a. For example, the first fluid control device 114a can be configured to be moved between a first position (e.g., a closed position, a fluidly disconnected position) in which fluid is inhibited from moving between the first catheter 120 and the first pressure source 106a and a second position (e.g., an open position, a fluidly connected position) in which fluid is permitted to move between the first catheter 120 and the first pressure source 106a. In some embodiments, the first connector 116a is a quick-release connector (e.g., a quick disconnect fitting) that enables rapid coupling/decoupling of the first catheter 120 and the first fluid control device 114a to/from the first pressure source 106a.

The second tubing assembly 110b can be configured to fluidly couple the second catheter 140 to a second pressure source 106b. In some embodiments the second pressure source 106b is separate from the first pressure source 106a, such as a separate syringe. In other embodiments, the second pressure source 106b can be omitted and the second tubing assembly 110b can be fluidly coupled to the first pressure source 106a in, e.g., series or parallel with the first pressure source 106a. The second tubing assembly 110b can include at least one second fluid control device 114b and at least one second connector 116b, each of which can be at least generally similar or identical in structure and/or function to the first fluid control device 114a and the first connector 116a, respectively.

In the illustrated embodiment, the mechanical thrombectomy catheter assembly 130 is positioned at least partially within the second lumen 123 with a portion thereof extending beyond the embolic protection device 136. The mechanical thrombectomy catheter assembly 130 can include a first or proximal actuation component 132, a second or distal actuation component 139, and one or more clot treatment devices 138. The proximal actuation component 132 (e.g., a proximal hub or handle) can be operably coupled to the clot treatment device 138 via an element shaft 142. Accordingly, moving the proximal actuation component 132, such as moving the proximal actuation component 132 in a proximal or distal direction, can cause a corresponding movement of the clot treatment device 138. In some embodiments, the element shaft 142 includes an atraumatic tip 146 defining a distalmost terminus of the mechanical thrombectomy catheter assembly 130. The distal actuation component 139 can be operably coupled to an additional delivery and/or deployment shaft 141 (which can also be referred to as a “delivery shaft,” an “intermediate shaft,” a “medial shaft,” a “third shaft,” a “catheter,” and/or the like) configured to extend through the second catheter 140 and over the element shaft 142 to constrain the clot treatment device 138. Accordingly, moving the distal actuation component 139, such as moving the distal actuation component 139 in the proximal or distal direction, can move the deployment shaft 141 relative to the clot treatment device 138 to, for example, deploy the clot treatment device 138 from within the deployment shaft 141. That is, the clot treatment device 138 can be positioned within, or at least partially within, the deployment shaft 141 such that the deployment shaft 141 maintains a collapsed or undeployed configuration/state of the clot treatment device 138, and the distal actuation component 139 can be withdrawn (e.g., proximally) over and/or relative to the element shaft 142 to deploy the clot treatment device 138. For example, the proximal actuation component 132 can be pinned in position and the distal actuation component 139 can be withdrawn relative to the proximal actuation component 132. The proximal actuation component 132 and the distal actuation component 139 can be moved in tandem and/or relative to one another, e.g., to insert the clot treatment device 138 through the embolic protection catheter assembly 128 and/or distally beyond the embolic protection device 136. The element shaft 142 can be positioned within and/or extend through the second catheter 140 (e.g., the lumen 123), such that all, or at least a portion, of the element shaft 142 is positioned radially inwardly from the second catheter 140. In at least some embodiments, for example, at least part of an outer surface of the element shaft 142 is in sliding contact with an interior or lumen of the second catheter 140.

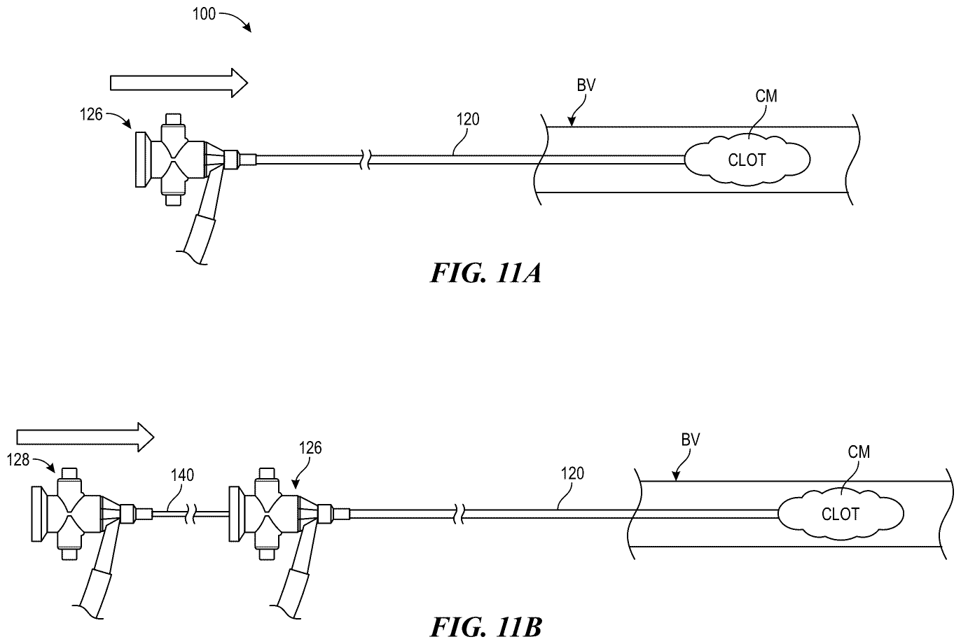

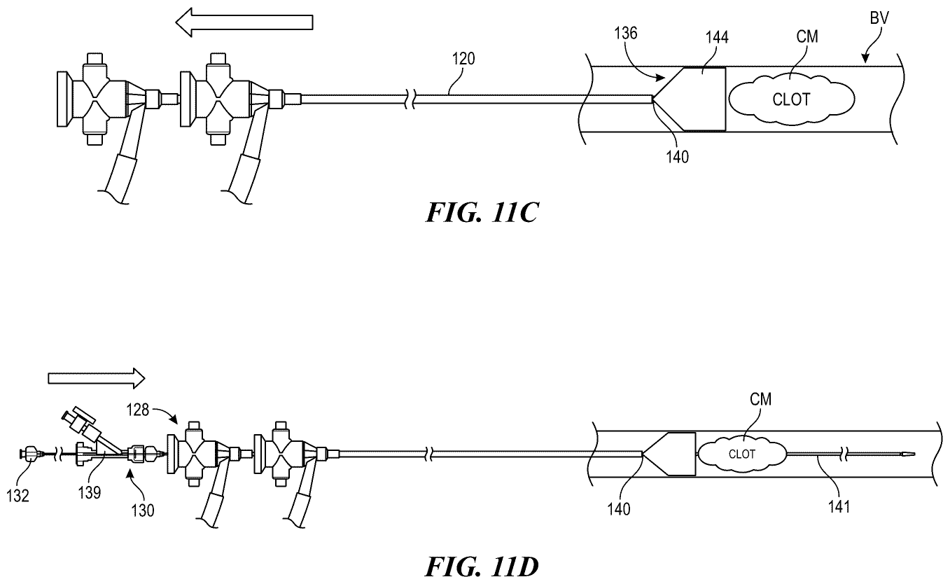

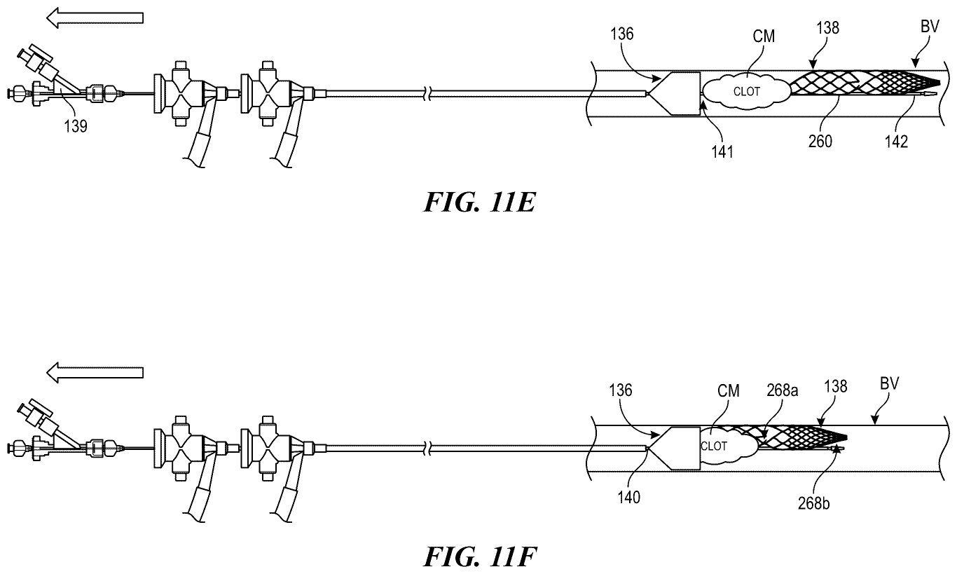

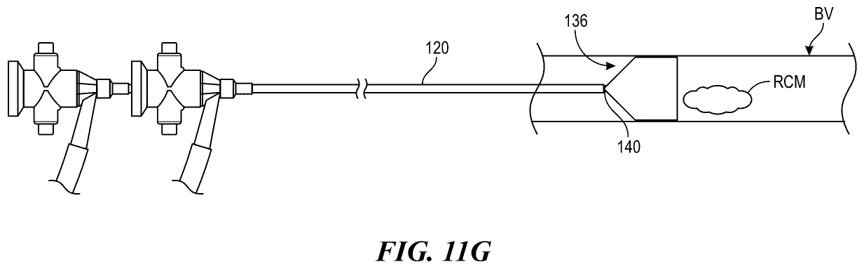

The distal actuation component 139 is shown fully withdrawn toward and/or into contact with the proximal actuation component 132 in FIG. 1, such that the clot treatment device 138 is deployed. Such deployment is described in further detail below with reference to FIGS. 11A-11G. In the illustrated embodiment, the distal actuation component 139 is a valve assembly including a connector 135 for attaching to one or more tubing sections, a fluid control device (which can be similar or identical to the fluid control devices 114a-b of FIG. 1), a pressure source (which can be similar or identical to the pressures source 106a and 106b of FIG. 1), etc., to, for example, facilitate aspiration through the deployment shaft 141.

As described in greater detail below with reference to FIGS. 11A-11G, during a procedure to treat clot material within a blood vessel of a patient, the system 100 can be inserted through the vasculature of the patient to proximate the clot material. In some embodiments, the system 100 is inserted through a separate introducer sheath that traverses the skin and tissue of the patient to provide an access site. In other embodiments, the introducer catheter assembly 126 serves as an introducer sheath for the embolic protection catheter assembly 128, and/or the mechanical thrombectomy catheter assembly 130. The funnel 144 can be deployed from the first catheter 120 proximal to the clot material, and the clot treatment device 138 can be deployed from the first catheter 120 (via, e.g., the deployment shaft 141) at least partially distal to the clot material. The clot treatment device 138 can engage the clot material to disrupt, capture, and/or core the clot material. The funnel 144 can capture any of the clot material that breaks off (e.g., embolizes) during the engagement of the clot treatment device 138 with the clot material and/or redirect all or a portion of the broken-off clot material into the second catheter 140—thereby inhibiting the clot material from flowing farther into the vasculature of the patient. In some embodiments, the embolic protection device 136 can inhibit or even prevent blood flow past, through, and/or around the embolic protection device 136 within the blood vessel. Finally, the embolic protection device 136 and the clot treatment device 138 can be withdrawn into the first catheter 120, and the clot treatment system 100 can be withdrawn from the patient.

In some embodiments, the first catheter 120 can be aspirated during, before, and/or after use of the clot treatment device 138. For example, when the first catheter 120 is positioned at a target treatment location proximate to the clot material, a user/operator can first close the first fluid control device 114a before generating a vacuum in the first pressure source 106a by, for example, withdrawing the plunger of a syringe coupled to the first connector 116a. In this manner, a vacuum is charged within the first pressure source 106a (e.g., a negative pressure is maintained) before the first pressure source 106a is fluidly connected to the first lumen 122 of the first catheter 120. To aspirate the first lumen 122, the user can open the first fluid control device 114a to fluidly connect the first pressure source 106a to the first catheter 120 and thereby apply or release the vacuum stored in the first pressure source 106a to the first lumen 122. Opening of the first fluid control device 114a instantaneously or nearly instantaneously applies the stored vacuum pressure to the first tubing assembly 110a and the first catheter 120, thereby generating a suction pulse throughout the first catheter 120 that can aspirate the clot material into the first catheter 120. In some embodiments, the vacuum from the first pressure source 106a is applied with the first fluid control device 114a in an open position (e.g., to provide continuous vacuum). That is, the user can generate the vacuum in the first pressure source 106a while the first fluid control device 114a is open (e.g., while the first pressure source 106a is fluidly connected to the first lumen 122 of the first catheter 120) to thereby aspirate the clot material while also simultaneously generating the vacuum, e.g., without or substantially without storing the vacuum in the first pressure source 106a. In other embodiments, the first catheter 120 can be continuously and/or intermittently aspirated via a vacuum pump (e.g., an electric vacuum pump) or other source of aspiration. Similarly, in some embodiments the second catheter 140 can be aspirated during, before, and/or after use of the clot treatment device 138. The second catheter 140 can be aspirated in a manner that is at least generally similar or identical to aspirating the first catheter 120, but using the second tubing assembly 110b instead of the first tubing assembly 110a. The first pressure source 106a or the second pressure source 106b can be used to store and/or apply the vacuum to the second tubing assembly 110b. Additionally, or alternatively, the first pressure source 106a, the second pressure source 106b, or another pressure source can be fluidly coupled to the connector 135 and used to aspirate a lumen of the deployment shaft 141.

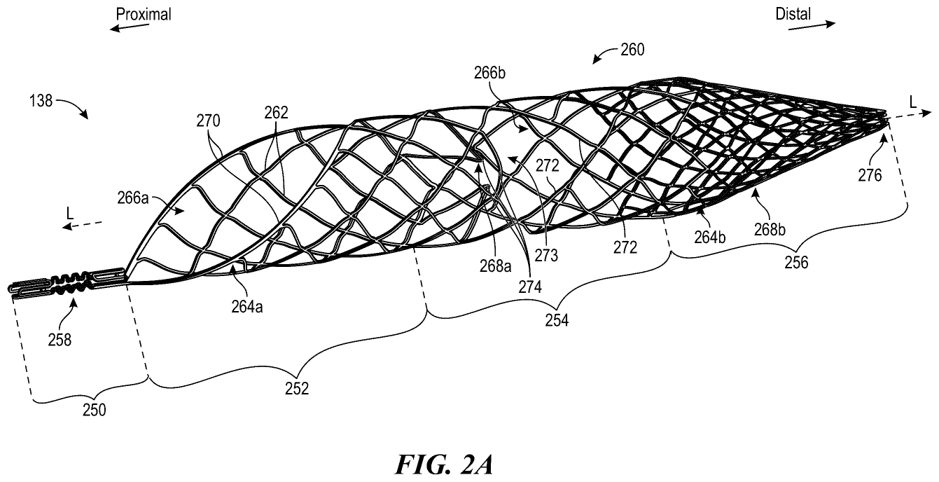

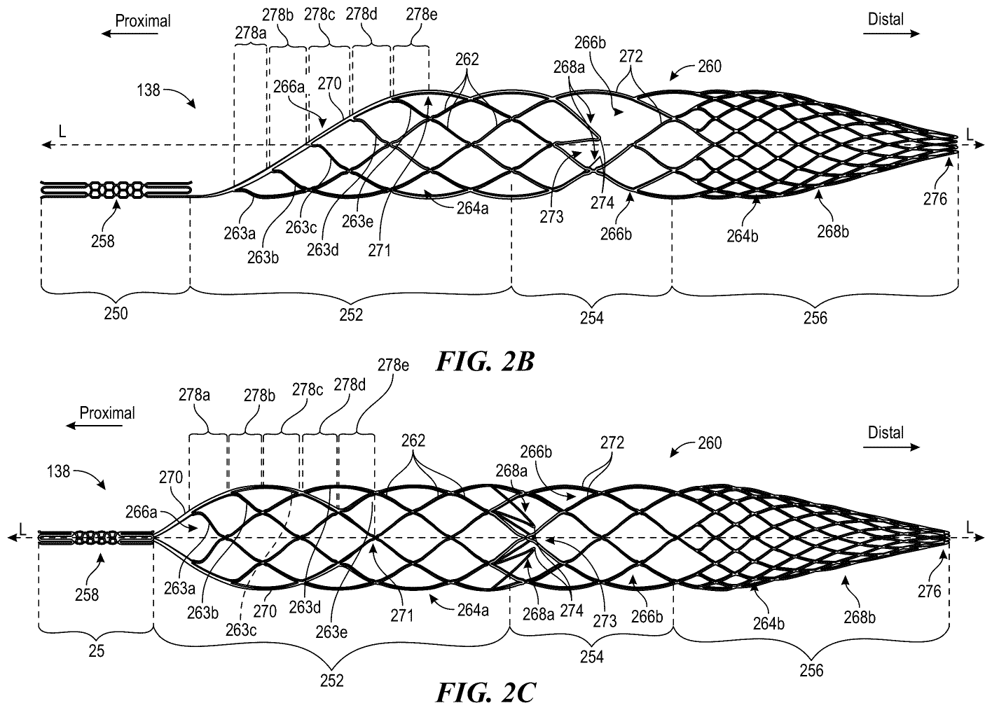

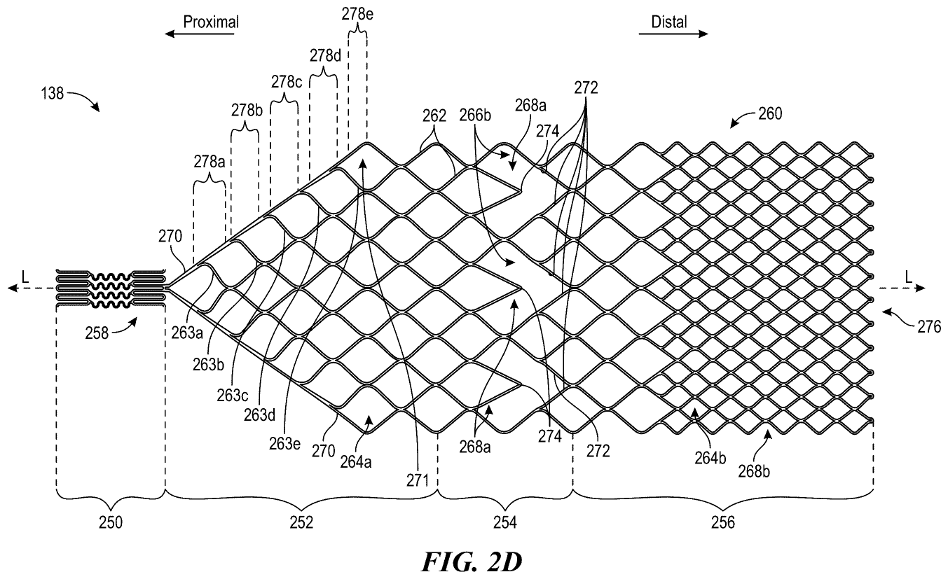

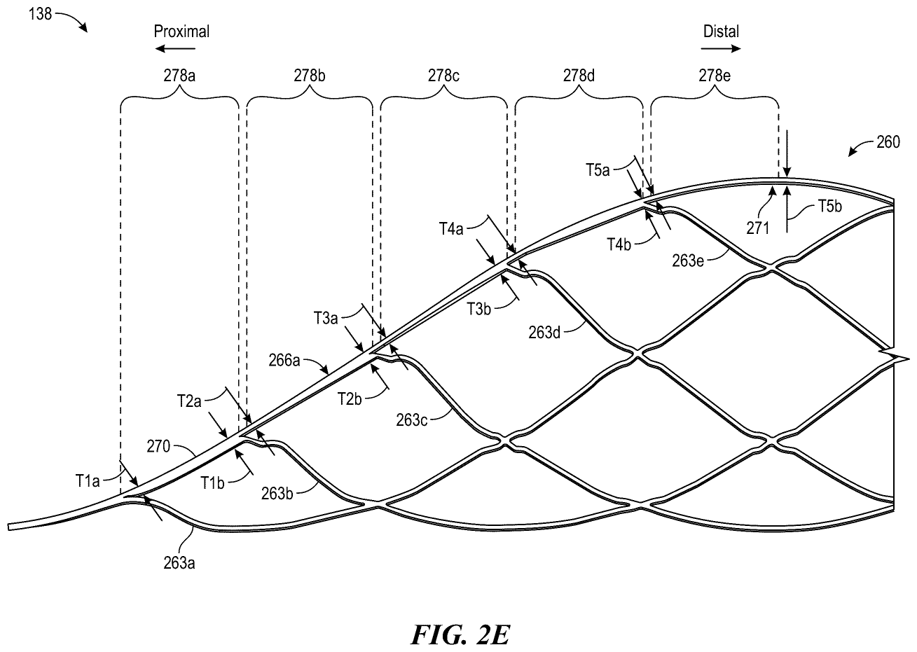

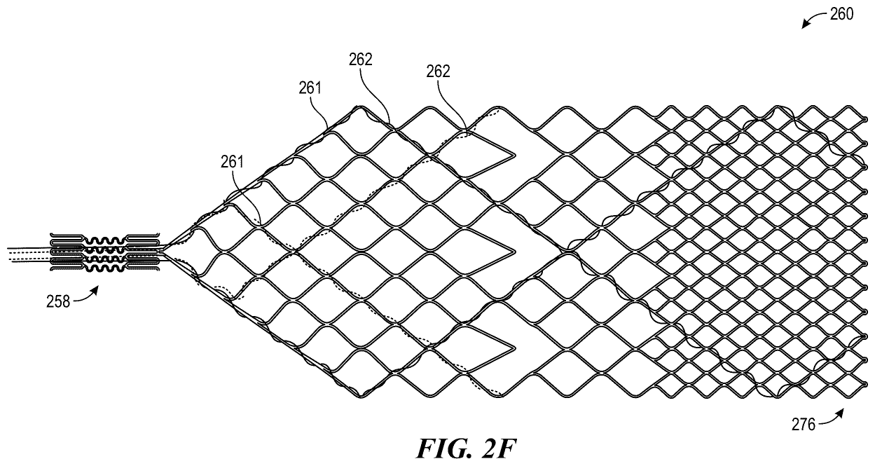

FIG. 2A is a perspective view of the clot treatment device 138 of FIG. 1 in accordance with embodiments of the present technology. FIGS. 2B and 2C are side and top views, respectively, of the clot treatment device 138 in accordance with embodiments of the present technology. In each of FIGS. 2A-2C, the clot treatment device 138 is in an expanded/deployed configuration in which the clot treatment device 138 has a generally cylindrical or tubular shape. FIG. 2D is a plan view (e.g., a cut and laid flat view) of the clot treatment device 138 in accordance with embodiments of the present technology. Referring to FIGS. 2A-2D together, the clot treatment device 138 can include a first region 250, a second region 252 distal of the first region 250, a third region 254 distal of the second region 252, and a fourth region 256 distal of the third region 254. The first region 250 can include a hub or coupling portion 258 configured to couple the clot treatment device 138 to the element shaft 142 (FIG. 1). The second region 252, the third region 254, and the fourth region 256 can together define an expandable clot removal element 260. The clot removal element 260 can be a single (e.g., unitary) laser cut metal (e.g., nitinol) element formed from a plurality of interconnected struts 262. The regions 252-256 can curve and/or extend at least partially around a central axis L.

The clot removal element 260 can be self-expanding and configured to transition from a collapsed or undeployed configuration to the expanded configuration shown in FIGS. 2A-2C. All or a subset of the struts 262 can be configured to self-expand when, e.g., the clot removal element 260 is positioned beyond (e.g., distally beyond) a distal terminus of the deployment shaft 141 (FIG. 1). The coupling portion 258 can be part of this single laser cut metal element, but can not self-expand with the clot removal element 260 (because, e.g., it is coupled to the element shaft 142). For example, the clot removal element 260 can be in the collapsed configuration when positioned within a larger delivery catheter, such as the first catheter 120, and the clot removal element 260 can expand to the expanded configuration when the element shaft 142 (FIG. 1) is advanced relative to the deployment shaft 141 (FIG. 1) so that the clot removal element 260 is no longer constrained by the deployment shaft 141. Retracting the element shaft 142 proximally within the deployment shaft 141 can return the clot removal element 260 to the collapsed configuration. In some embodiments, the clot removal element 260 and/or one or more of the other clot treatment devices described herein can include some features that are generally similar or identical, and can function in a generally similar or identical manner, to any of the clot removal elements described in detail U.S. patent application Ser. No. 17/072,909, titled “SYSTEMS, DEVICES, AND METHODS FOR TREATING VASCULAR OCCLUSIONS,” filed Oct. 16, 2020; U.S. patent application Ser. No. 17/125,397, titled “DEVICES AND METHODS FOR TREATING VASCULAR OCCLUSION,” filed Dec. 17, 2020; and/or U.S. Pat. No. 10,709,471, titled “METHODS AND APPARATUS FOR TREATING EMBOLISM,” and filed Apr. 10, 2018; and U.S. patent application Ser. No. 18/349,674, titled “MECHANICAL THROMBECTOMY ASSEMBLY WITH EMBOLIC PROTECTION, AND ASSOCIATED DEVICES, SYSTEMS, AND METHODS,” and filed Jul. 8, 2023; each of which is hereby incorporated by reference in its entirety.

The struts 262 can define one or more cells 264 (individually identified as first cells 264a and second cells 264b), one or more mouths 266 (individually identified as a first or proximal mouth 266a and one or more second or distal mouths 266b), and one or more relief features 268 (individually identified as one or more first relief features 268a and a second relief feature 268b). The first relief features 268a can be referred to as proximal or middle relief features, and the second relief feature 268b can be referred to as a distal relief feature. In the illustrated embodiment, for example, the second region 252 includes one or more of the first cells 264a and the fourth region 256 includes one or more of the second cells 264b. All or subset of the second cells 264b can be smaller (e.g., smaller area, smaller width, smaller diameter, etc.) than all or a subset of the first cells 264a. In other embodiments, however, all or subset of the second cells 264b can be larger (e.g., larger area, larger width, larger diameter, etc.) than all or a subset of the first cells 264a.

The second region 252 can further include one or more leading edge portions 270 that define the first or proximal mouth 266a. As best seen in FIGS. 2B-2D, one or more of the leading edge portions 270 can include one or more strut segments 278 (individually identified as first through fifth segments 278a-c; for illustrative clarity, these are only labeled for one of the leading edge portions 270). Each of the strut segments 278 can be defined or extend between two or more proximal struts 263 (individually identified as first through fifth proximal struts 263a-e; for illustrative clarity, these are only labeled for one of the leading edge portions 270). In the illustrated embodiment, for example, (i) the first segment 278a extends between the first proximal strut 263a and the second proximal strut 263b, (ii) the second segment 278b extends between the second proximal strut 263b and the third proximal strut 263c, (iii) the third segment 278c extends between the third proximal strut 263c and the fourth proximal strut 263d, and (iv) the fourth segment 278d extends between the fourth proximal strut 263d and the fifth proximal strut 263c. The fifth segment 278e extends between the fifth proximal strut 263e and an apex or distal terminus 271 of the leading edge portion 270.

FIG. 2E is an enlarged side view of one of the leading edge portions 270. As best seen in FIG. 2E, each of these segments 278 can have a thickness that cascades or varies over its length. In the illustrated embodiment, for example, the thickness of one or more of the segments 278 increases in a proximal-to-distal direction over the length of each individual segment 278 but can decrease at the transition between adjacent (e.g., immediately adjacent) segments. More specifically, (i) the first segment 278a can have a first primary thickness T1a at the first proximal strut 263a and a first secondary thickness T2a at the second proximal strut 263b that is greater than the first primary thickness T1a, (ii) the second segment 278b can have a second primary thickness T2a at the second proximal strut 263b and a second secondary thickness T2b at the third proximal strut 263c that is greater than the second primary thickness T2a, (iii) the third segment 278c can have a third primary thickness T3a at the third proximal strut 263c and a third secondary thickness T3b at the fourth proximal strut 263d that is greater than the third primary thickness T3a, and (iv) the fourth segment 278d can have a fourth primary thickness T4a at the fourth proximal strut 263d and a fourth secondary thickness T4b at the fifth proximal strut 263e that is greater than the fourth primary thickness T4a. The fifth segment 278e can have a fifth primary thickness T5a at the fifth proximal strut 263c and a fifth secondary thickness T5b at the apex terminus 271 that is less than the fifth primary thickness T5a. The first primary thickness T1a can be greater than the second primary thickness T2a, the second primary thickness T2a can be greater than the third primary thickness T3a, the third primary thickness T3a can be greater than the fourth primary thickness T4a, the fourth primary thickness T4a can be greater than the fifth primary thickness T5a, and the fifth primary thickness T5a can be greater than the fifth secondary thickness T5b. The first secondary thickness T1b can be greater than the second secondary thickness T2b, the second secondary thickness T2b can be greater than the third secondary thickness T3b, the third secondary thickness T3b can be greater than the fourth secondary thickness T4b, the fourth secondary thickness T4b can be greater than the fifth primary thickness T5a, and the fifth primary thickness T5a can be greater than the fifth secondary thickness T5b. Accordingly, the thicknesses associated with the segments 278 can be defined as follows:

$$T 1 b > T 1 a$$

$$T 1 b > T 2 b > T 3 b > T 4 b > T 5 a > T 5 b$$

$$T 1 a > T 2 a > T 3 a > T 4 a > T 5 a > T 5 b$$

The varying thicknesses of the segments 278 can allow the leading edge portions 270 to preferentially collapse or close in a distal-to-proximal direction. For example, in response to a compressive force on the clot removal element 260, the fifth segment 278e can collapse or flex radially inwardly, followed (e.g., one-by-one, in sequence, etc.) by the fourth segment 278d, the third segment 278c, the second segment 278b, and the first segment 278a. This is expected to allow the proximal mouth 266a to remain at least partially open, even in small diameter vessels, and, in turn, allow the clot removal element 260 to be used to remove clot material from those small diameter vessels.

Returning to FIGS. 2A-2D, the third region 254 can include one or more intermediate or medial edge portions 272 that define the one or more second or distal mouths 266b. The one or more second mouths 266b can be longitudinally offset from (e.g., positioned distal to) the first mouth 266a. Also in the third region 254, one or more of the struts 262 can terminate at one or more junctions 274 to define the first relief features 268a. In the illustrated embodiment, for example, pairs of the struts 262 in the third region 254 join at each of the junctions 274 to define a corresponding one of the first relief features 268a. As best seen in FIGS. 2A-2C, one or more of the first relief features 268a can be angled inwardly (e.g., toward the central axis L) and, accordingly, can be configured to contain or hold clot material ingested through the first mouth 266a within at least the second region 252. In some embodiments, the first relief features 268a can be configured to distribute captured clot material within the clot removal element 260 to, e.g., inhibit or prevent the captured clot material from accumulating at the distal end of the clot removal element 260. For example, as shown in FIGS. 2A-2C, the junctions 274 can be spaced apart from one another to define a central passage 273 between the first relief features 268a. A first portion of the clot material captured within the clot removal element 260 can pass distally through the central passage 273 while a second portion of the captured clot material can be retained proximal to the first relief features 268a, distributing captured clot material longitudinally within the clot removal element 260.

When a force on the first relief features 268a exceeds a corresponding first threshold, the first relief features 268a can be configured to bend or flex outwardly (to, e.g., rotate or otherwise move away from the central axis L). As described previously herein, the struts 262 can be formed from a shape-memory material such as Nitinol and, accordingly, in at least some embodiments the first relief features 268a can be shape set to the inwardly-angled orientation. Accordingly, in at least some embodiments, the first threshold associated with the first relief features 268a can be based on one or more of the shape set properties of the first relief features 268a. Additionally, or alternatively, the first threshold can be based, at least in part, on the thickness, length, angle, and/or other properties of the first relief features 268a. In these and/or other embodiments, the first threshold can be less than the yield strength, the ultimate strength, and/or the fracture strength of the struts 262 themselves and/or one or more other portions of the clot treatment device 138. Accordingly, in response to increasing amounts of force on the clot treatment device 138, the first relief features 268a are expected to bend/flex before other portions of the clot treatment device 138 yield, break, or otherwise fail.

In the fourth region 256, the struts 262 and/or the second cells 264b can be tapered radially inwardly toward the central axis L and/or a distal terminus 276 of the clot removal element 260. This radially inward taper of the struts 262 and/or the second cells 264b in the fourth region 256 can define the second relief feature 268b. When a force on the second relief feature 268b exceeds a corresponding second threshold, the second relief feature 268b can be configured to bend or flex outwardly (e.g., away from the central axis L) to open the distal terminus 276 of the clot removal element. That is, the generally conical shape of the fourth region 256 can deflect away from the central axis L to allow clot material to pass through the distal terminus when the second threshold force is exceeded. As described previously herein, the struts 262 can be formed from a shape-memory material such as Nitinol and, accordingly, in at least some embodiments the second relief feature 268b can be shape set to the inwardly-tapered configuration. Accordingly, in at least some embodiments, the second threshold can be based on one or more of the shape set properties of the second relief feature 268b. Additionally, or alternatively, the second threshold can be based, at least in part, on the thickness, length, angle, and/or other properties of the second relief feature 268b. In these and/or other embodiments, the second threshold can be less than the yield strength, the ultimate strength, and/or the fracture strength of the struts 262 themselves and/or one or more other portions of the clot treatment device 138. Accordingly, in response to increasing amounts of force on the clot treatment device 138, the second relief feature 268b is expected to bend/flex before other portions of the clot treatment device 138 yield, break, or otherwise fail. The distal terminus 276 can be an unconstrained and/or free end of the clot removal element 260. In at least some embodiments, for example, the distal terminus 276 is not connected to the element shaft 142 (FIG. 1) and/or one or more catheters or shafts.

Referring again to FIGS. 2A-2D, during a clot treatment procedure the clot removal element 260 can mechanically engage clot material within a patient's blood vessel to disrupt, capture, and/or core the clot material. For example, the clot removal element 260 can be deployed distal to the clot material and moved proximally toward and/or at least partially through the clot material. As the clot removal element 260 is moved proximally, the first mouth 266a can core or capture at least a portion of the clot material and direct the cored/captured clot material into the clot removal element 260. The one or more second mouths 266b can, due to their longitudinally offset position from the first mouth 266a, core or capture additional and/or other clot material than the first mouth 266a. For example, as the clot removal element 260 is moved proximally through clot material within a patient's blood vessel, the first mouth 266a can core/capture a first portion of the clot material and the one or more second mouths 266b can core/capture a second portion of the clot material different than the first portion. The cells 264 defined by the struts 262 can be sized and/or otherwise configured to generally retain the capture clot material within the clot removal element 260 while allowing blood and/or other fluids within a patient's blood vessel to pass through the clot removal element 260. As clot material is captured by the first mouth 266a and accumulates within the second region 252, the accumulated clot material can apply an increasing force on the first relief features 268a. This force on the first relief features 268a can also be increased when, for example, a user tries to withdraw the clot removal element 260 into the funnel 144 (v 1) and/or the deployment shaft 141 while the clot removal element 260 contains clot material. When the magnitude of that force exceeds the first threshold associated with the first relief features 268a, the first relief features 268a can bend or flex outwardly (e.g., away from the central axis L) and allow at least a portion of the clot material within the second region 252 to move distally past the first relief features 268a, through the third region 254, and/or into the fourth region 256. This can, in turn, reduce the total force on the clot removal element 260 and inhibit or even prevent the clot removal element 260 from yielding, breaking, or otherwise failing. As clot material accumulates within the fourth region 256, either after the first relief features 268a release a portion of the accumulated clot material and/or as the second mouths 266b capture additional clot material, the accumulated clot material can apply an increasing force on the second relief feature 268b. This force on the second relief feature 268b can also be increased when, for example, a user tries to withdraw the clot removal element 260 into the funnel 144 (FIG. 1) and/or the deployment shaft 141 while the clot removal element 260 contains clot material. When the magnitude of that force exceeds the second threshold associated with the second relief feature 268b, the second relief feature 268b can be configured to bend or flex outwardly (e.g., away from the central axis L) to expand or open the distal terminus 276 and release clot material from within the fourth region 256. This can, in turn, reduce the total force on the clot removal element 260 and inhibit or even prevent the clot removal element 260 from yielding, breaking, or otherwise failing.

FIG. 2F is another plan view of the clot treatment device 138 in the pre-shaped configuration. In some embodiments, the clot removal element 260 can include one or more visualization features 261 configured to improve imaging/visualization of the clot removal element 260, e.g., when the clot removal element 260 is positioned within a patient. In the illustrated embodiment, the visualization features 261 include one or more radio-opaque wires wound/wrapped around individual ones of the struts 262, e.g., through one or more of the mouths and/or cells defined by the struts 262. In some embodiments, the visualization features 261 comprise multiple wires that are wrapped around the struts 262. These wires can extend in a direction from the coupling portion 258 toward and/or to the distal terminus 276 and, in at least some embodiments, return toward and/or to the coupling portion 258 to, e.g., have a helical shape about the clot removal element 260. In some embodiments, the visualization features 261 include two wires wrapped in such a manner. The wires can be formed from platinum and/or one or more other suitable materials. In these and/or other embodiments, the visualization features 261 can include one or more dyes, coatings, and/or other suitable visualization features.

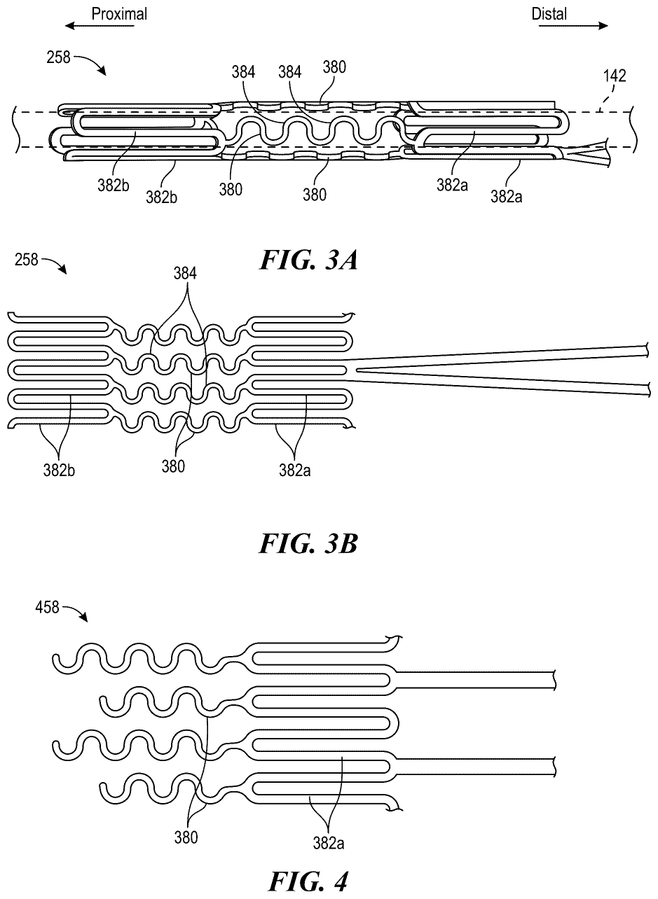

FIG. 3A is a side view of the coupling portion 258 of the clot treatment device 138, and FIG. 3B is a plan view (e.g., a cut and laid flat view) of the coupling portion 258 in accordance with embodiments of the present technology. Referring to FIGS. 3A and 3B together, the coupling portion 258 can include one or more attachment struts 380 and one or more terminal struts 382 (individually identified as distal terminal struts 382a and proximal terminal struts 382b). One or more of the attachment struts 380 can have an undulating (e.g., wavy) or other curved shape that defines one or more strut portions 384 that extend perpendicularly, or at least generally perpendicularly, to a length of the element shaft 142. One or more of the terminal struts 382 can extend parallel, or at least generally parallel, to the length of the element shaft 142 (shown in dashed line in FIG. 3A). In the illustrated embodiment, one or more of the attachment struts 380 extend between or couple one or more of the distal terminal struts 382a to one or more of the proximal terminal struts 382b. In other embodiments, one or more of the distal and/or proximal terminal struts 382a and/or 382b can be omitted.

The coupling portion 258, including all or a subset of the attachment struts 380 and/or the terminal struts 382, can be coupled (via, e.g., adhesive and/or polymer reflow) to the element shaft 142. When the clot removal element 260 (FIGS. 1-2C) is in use, such as while clot material accumulates within the clot removal element 260, this coupling between the coupling portion and the element shaft 142 can be subject to one or more forces and/or loads, including e.g., tensile forces, compressive loads, torsional loads, rotational forces, and/or bending in, e.g., the proximal or distal direction. In some instances, these forces/loads can cause the element shaft 142 to stretch or flex. In response to these forces, one or more of the attachment struts 380 can elongate and distribute these forces more evenly along both the element shaft 142 and/or through the attachment struts 380 themselves, which can inhibit or even prevent weak points from developing. In at least some embodiments, for example, the coupling portion 258 can bend or flex with the element shaft 142, inhibiting or even preventing relative movement between these components that can stress the attachment/bond therebetween.

The curved shape of the attachment struts 380 can provide an increased surface area over which the attachment struts 380 can be coupled to the element shaft 142 and, accordingly, can improve the engagement of the coupling portion 258 with the element shaft 142. The length and/or amplitude of the undulating shape of the attachment struts 380 can be tuned to emphasize elongation or to emphasize surface area based on, e.g., the catheter size. Similarly, the terminal struts 382 can provide an increased surface area over which the attachment struts 380 can be coupled to the element shaft 142. The tangential strut portions 384 can also improve the coupling portion's engagement with the element shaft 142. For example, when the attachment struts 380 are adhered to the element shaft 142 using, e.g., a bond material, one or more of the tangential strut portions 384 can engage the bond material and serve as clamps that provide increasing engagement as the tensile force increases.

FIG. 4 is a plan view (e.g., cut and laid flat view) of another coupling portion 458 in accordance with embodiments of the present technology. The coupling portion 458 can include one or more features that are at least generally similar or identical in structure and/or function to the coupling portion 258 of FIGS. 3A and 3B. For example, the coupling portion 458 includes one or more of the attachment struts 380 and one or more of the distal terminal struts 382a. However, compared to the coupling portion 258 (FIGS. 3A and 3B), the coupling portion 458 omits the proximal terminal struts 382b.

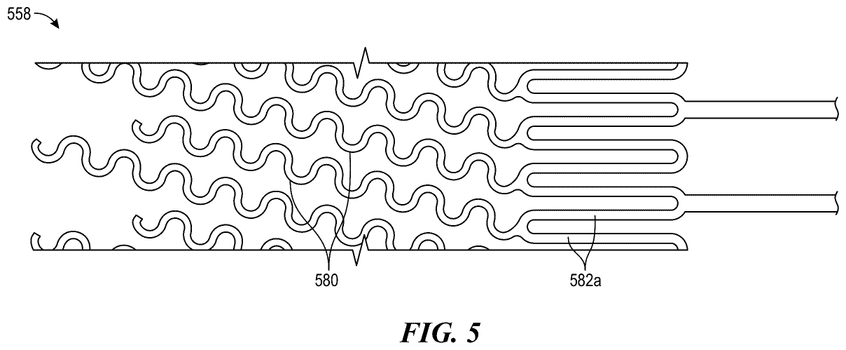

FIG. 5 is a plan view (e.g., cut and laid flat view) of another coupling portion 558 in accordance with embodiments of the present technology. The coupling portion 458 can include one or more features that are at least generally similar or identical in structure and/or function to the coupling portion 458 of FIG. 4. For example, the coupling portion 558 includes one or more attachment struts 580 and one or more distal terminal struts 582a. However, compared to the coupling portion 458 (FIG. 4), the attachment struts 580 extend proximally and at an angle from the distal terminal struts 582a. Accordingly, when the coupling portion 558 is coupled to the element shaft 142 (FIG. 1), the attachment struts 580 can extend helically around the element shaft 142. This helical arrangement of the distal terminal struts 582a can add additional dynamic tensile translation. For example, when force is applied to the coupling portion 558, one or more of the attachment struts 580 can stretch and/or be drawn/pressed inwardly against the element shaft 142, providing or increasing an inward clamp force against the element shaft 142. This increases the hold of the coupling portion 558 on the element shaft 142 while also inhibiting or preventing the formation of weak points, as described above with reference to FIGS. 3A and 3B.

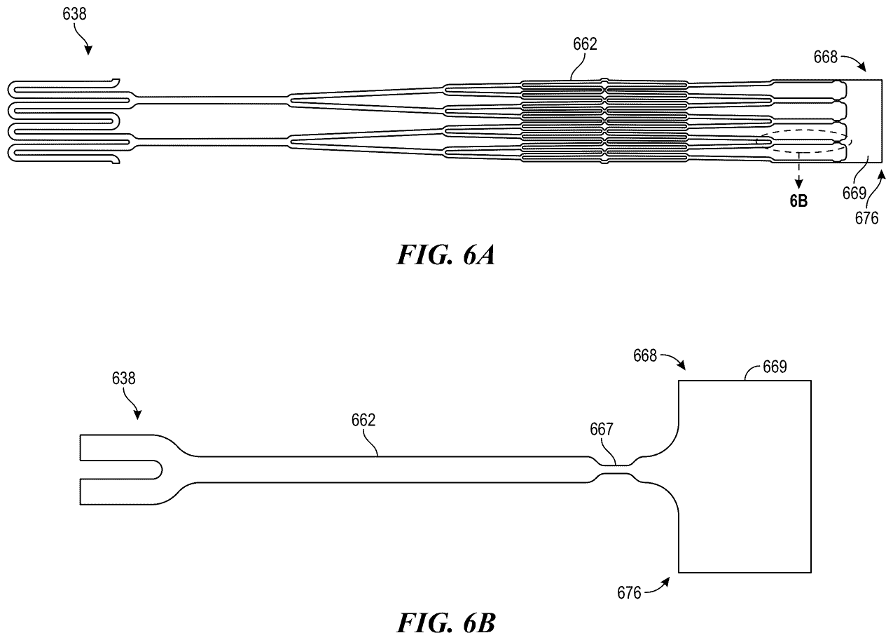

FIG. 6A is a plan view (e.g., a cut and laid flat view) of a clot treatment device 638 configured in accordance with additional embodiments of the present technology. FIG. 6B is an enlarged view of region 6B in FIG. 6A. The clot treatment device 638 can be at least generally similar in structure and/or function to one or more of the other clot treatment devices described herein. For example, the clot treatment device 638 can include a relief feature 668 (e.g., a distal relief feature) that is generally similar to the second relief feature 268b of the clot treatment device 138 of FIGS. 2A-2C. For example, the clot treatment device 638 can include one or more struts 662, and the relief feature 668 can be positioned at or define a distal terminus 676 of the clot treatment device 638. However, in the illustrated embodiment, the relief feature 668 includes a collar or cuff 669 and, as best seen in FIG. 6B, the cuff 669 can be coupled to one or more of the struts 662 by one or more fuses or frangible strut sections 667. In the illustrated embodiment, the frangible strut sections 667 are narrowed portions of the struts 662 that are configured to break when a force on the frangible strut sections 667 equals or exceeds a corresponding threshold. In other embodiments, one or more of the frangible strut sections 667 can be perforated or otherwise configured to break when the force equals or exceeds the threshold. The threshold can be less than the yield strength, the ultimate strength, and/or the fracture strength of the struts 662 themselves and/or one or more other portions of the clot treatment device 638.

The cuff 669 can hold the distal terminus 676 closed to inhibit or even prevent clot material within the clot treatment device 638 from moving distally out from the interior of the clot treatment device 638. However, the cuff 669 can be subject to increasing magnitudes of force as clot material accumulates within the clot treatment device 638. When the force on the cuff 669 exceeds the threshold, all or a subset of the frangible strut sections 667 can be configured to break, allowing the distal terminus 676 of the clot treatment device 638 to open and release captured clot material, thereby reducing the force and/or load on other portions of the clot treatment device 638. Accordingly, in response to increasing amounts of force on the clot treatment device 638, the frangible strut sections 667 are expected to break before other portions of the clot treatment device 638 yield, break, or otherwise fail.

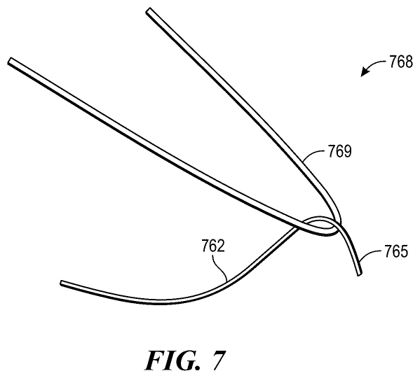

FIG. 7 is a perspective view of another relief feature 768 configured in accordance with additional embodiments of the present technology. The relief feature 768 can include one or more struts 762 that define a hook portion 765 and a loop 769 of, e.g., one or more of the other struts 762. The hook portion 765 can engage the loop 769, as shown in FIG. 7, to retain clot material within a clot treatment device. However, as hook portion 765 and the loop 769 can be subject to increasing magnitudes of force as clot material accumulates within a clot treatment device incorporating the relief feature 768. When the force on the hook portion 765 and/or the loop 769 exceeds a threshold, the hook portion 765 can bend, flex, or otherwise disengage the loop 769 to open the clot treatment device and release captured clot material to thereby reduce the force and/or load on other portions of the clot treatment device.

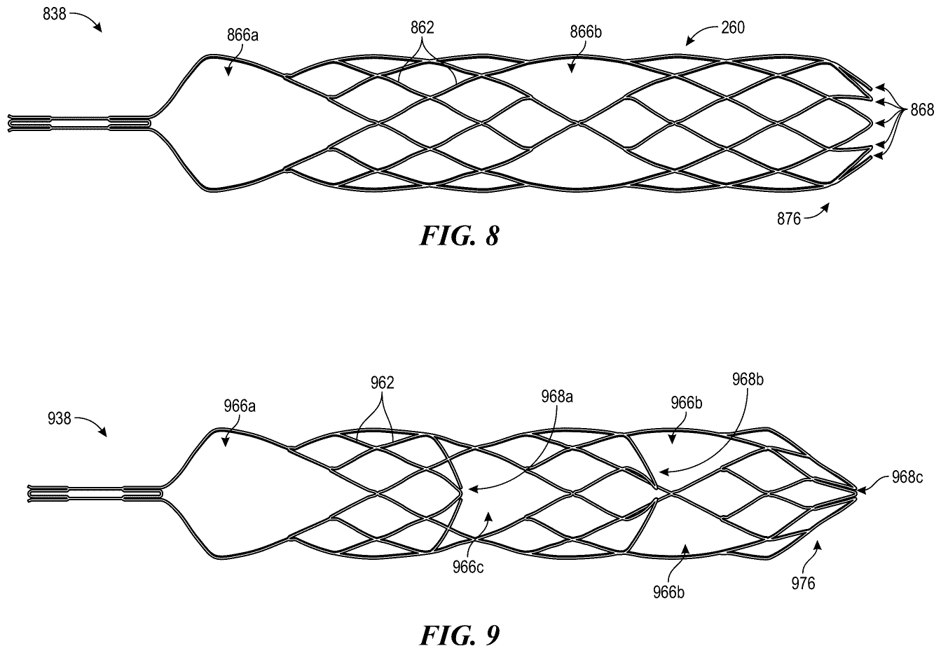

FIG. 8 is a top view of another clot treatment device 838 configured in accordance with embodiments of the present technology. At least some aspects of the clot treatment device 838 can be at least generally similar or identical in structure and/or function to one or more of the clot treatment devices described previously herein. For example, the clot treatment device 838 can be formed from one or more struts 862 and define a first or proximal mouth 866a and one or more second or distal mouths 866b that can be spaced longitudinally apart (e.g., distally from) the proximal mouth 866a. The clot treatment device 838 can also include one or more relief features 868, individual ones of which can be at least generally similar or identical in structure and/or function to the first relief features 268a of FIG. 2A. However, compared to the first relief features 268a, the relief features 868 can define a distal terminus 876 of the clot treatment device 838 and, e.g., the clot treatment device 838 can omit any other relief features.

FIG. 9 is a top view of another clot treatment device 938 configured in accordance with embodiments of the present technology. At least some aspects of the clot treatment device 938 can be at least generally similar or identical in structure and/or function to one or more of the clot treatment devices described previously herein. For example, the clot treatment device 938 can be formed from one or more struts 962 and define a first or proximal mouth 966a and one or more second or distal mouths 966b that can be spaced longitudinally apart (e.g., distally from) the proximal mouth 966a. Additionally, the clot treatment device 938 can define one or more third or medial mouths 966c positioned between the proximal and distal mouths 966a, 966b. The clot treatment device 838 can also include one or more relief features 968 (individually identified as one or more first relief features 968a, one or more second relief features 968b, and one or more third relief features 968c) individual ones of which can be at least generally similar or identical in structure and/or function to the first relief features 268a of FIG. 2A. The one or more first relief features 968a can be positioned at least partially between the proximal and medial mouths 966a, 966c. The one or more second relief features 968b can be positioned at least partially between the medial and distal mouths 966c, 966b. The one or more third relief features 968c can define a distal terminus 976 of the clot treatment device 938.

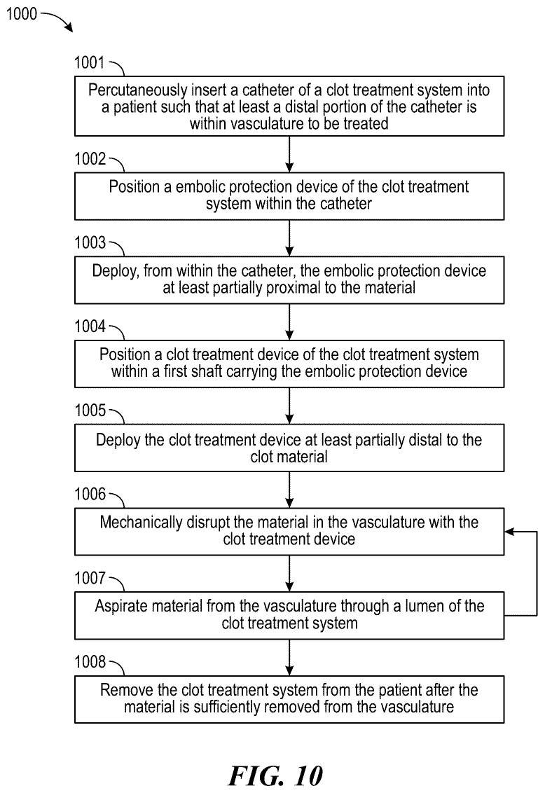

FIG. 10 is a flow diagram of a process or method 1000 for removing clot material from the vasculature of a patient using a clot treatment system in accordance with embodiments of the present technology. Although some features of the method 1000 are described in the context of the clot treatment system 100 shown in FIG. 1 for illustration, one skilled in the art will readily understand that the method 1000 can be carried out using other suitable systems and/or devices described herein, including any of the other clot removal devices described herein. FIGS. 11A-11G are side views of the clot treatment system 100 of FIG. 1 during different stages of the method 1000, in accordance with embodiments of the present technology. Individual ones of FIGS. 11A-11G are described below with reference to one or more blocks 1001-1008 of the method 1000.

At block 1001, the method 1000 can include percutaneously inserting a catheter of a clot treatment system into a patient such that at least a distal portion of the catheter is positioned within a blood vessel to be treated. For example, FIG. 11A shows introducer catheter assembly 126, including the first catheter 120, inserted through the skin of a patient and into the blood vessel BV, including clot material CM to be treated. In some instances, the clot material CM can substantially occlude the blood vessel BV, such as shown in FIG. 11A, or can partially occlude the blood vessel BV. The blood vessel BV can comprise a peripheral artery of a human patient, and the clot material CM can comprise a thromboembolism therein. Accordingly, the method 1000 can be a method for treating acute limb ischemia (ALI), acute visceral ischemia, chronic limb ischemia (CLI), and/or the like.

At block 1002, the method 1000 can include positioning an embolic protection device of the clot treatment system within the catheter. For example, FIG. 11B shows the embolic protection catheter assembly 128, including the second catheter 140 positioned within and/or extending through the introducer catheter assembly 126 and the first catheter 120 thereof. The second catheter 140 can carry the embolic protection device 136 (FIG. 1) so that both can be advanced distally through the first catheter 120 toward the clot material CM.

At block 1003, the method 1000 can include deploying, from within the catheter, the embolic protection device at least partially proximal to the clot material in the blood vessel. For example, FIG. 11C shows the embolic protection device 136 including the funnel 144 deployed/expanded within the blood vessel BV, with at least a portion of the funnel 144 positioned proximally of the clot material CM. The embolic protection device 136 can be deployed/expanded by retracting the first catheter 120 (e.g., proximally and/or over the second catheter 140) to uncover the embolic protection device 136. When expanded/deployed, the embolic protection device 136 can span all or a portion of the width/diameter of the blood vessel BV and block or substantially block blood flow through the blood vessel BV.

At block 1004, the method 1000 can include positioning a clot treatment device within the first shaft of the embolic protection device. For example, FIG. 11D shows the deployment shaft 141 of the mechanical thrombectomy catheter assembly 130 positioned within and advanced through the second catheter 140 of the embolic protection catheter assembly 128. The deployment shaft 141 can cover and/or constrain the clot treatment device 138 (FIGS. 1-2F). Accordingly, the clot treatment device 138 can be advanced together with the deployment shaft 141 while constrained therein. In some embodiments, at least a portion of the deployment shaft 141 can extend distally beyond the second catheter 140 and/or the clot material CM, as shown in FIG. 11D. During insertion, the proximal actuation component 132 and the distal actuation component 139 can be spaced apart from one another. For example, as described above with reference to FIG. 1, the proximal actuation component 132 and/or the element shaft 142 (FIG. 1) can be pinned in position relative to the distal actuation component 139 such that the clot treatment device 138 (FIG. 1) is constrained within the deployment shaft 141.

At block 1005, the method 1000 can include deploying the clot treatment device at least partially distal to the clot material in the blood vessel. For example, FIG. 11E shows the clot treatment device 138 deployed/expanded within the blood vessel BV, with at least a portion of the clot treatment device 138 positioned distal to the clot material CM. The clot treatment device 138 can be deployed/expanded by retracting the deployment shaft 141 (e.g., proximally and/or over the element shaft 142) to uncover the clot removal element 260 carried thereby. To withdraw the deployment shaft 141, the distal actuation component 139 can be withdrawn over the element shaft 142 toward the proximal actuation component 132, decreasing the distance between the distal actuation component 139 and the proximal actuation component 132, e.g., until the distal actuation component 139 contacts the proximal actuation component 132 as shown in FIG. 11E. In some embodiments, deploying the clot treatment device includes centering the clot treatment device within the blood vessel. For example, the clot treatment device 138 can be configured to self-center in response to, e.g., contact with the blood vessel BV and/or one or more forces on the clot treatment device 138 that cause the clot treatment device 138 to deploy/expand.

At block 1006, the method 1000 can include mechanically disrupting the clot material in the blood vessel with the clot treatment device. For example, FIG. 11F shows the clot treatment device 138 (e.g., the clot removal element 260, shown in FIG. 11E) retracted (e.g., proximally) to capture at least a portion of the clot material CM (via, e.g., the first mouth 266a and/or one or more of the second mouths 266b; FIGS. 2A-2D). The clot treatment device 138 can be retracted through/against the clot material CM toward the embolic protection device 136 and/or at least partially into the interior of the funnel 144. The embolic protection device 136 (e.g., the funnel 144) can be positioned proximal to the clot material CM and configured to inhibit or even prevent blood flow (in the direction shown by the arrow B) through the blood vessel BV. In some aspects of the present technology, blocking blood flow through the blood vessel BV via the embolic protection device 136 can inhibit portions (e.g., fragments) of the clot material CM from migrating downstream farther into the vasculature of the patient. Additionally, the embolic protection device 136 can direct the clot removal element 260 and the captured clot material contained within the clot removal element 260 into the second catheter 140. Accordingly, the embolic protection device 136 is expected to capture and/or direct all, or at least a portion, of the clot material CM into the second catheter 140. In some embodiments, after the clot material CM is sufficiently disrupted, block 1006 can include removing the clot treatment device from the patient, e.g., by retracting (e.g., proximally) one or both of the deployment shaft 141 and the element shaft 142 into and/or through the second catheter 140 and/or the first catheter 120. In these and/or other embodiments, the clot removal element 260 can be reintroduced one or more times as needed to remove additional clot material in a manner at least generally similar or identical to blocks 1004 and/or 1005.

In some embodiments, mechanically disrupting the clot material CM can include bringing clot material into contact with one or more of the relief features 268. For example, retracting the clot removal element 260 proximally to capture clot material CM within the clot removal element 260 can drive at least some clot material CM against one or more of the first relief features 268a and/or the second relief feature 268b. If the force on the first relief features 268a exceeds a first threshold then, as described previously with reference to FIGS. 2A-2D, one or more of the first relief features 268a can bend or flex outwardly and allow clot material CM within the clot removal element 260 to move further distally through the clot removal element 260 until, e.g., the clot material CM contacts the second relief feature 268b. If the force on the second relief feature 268b exceeds a second threshold then, as described previously with reference to FIGS. 2A-2D, the second relief feature 268b can bend or flex outwardly and allow clot material CM within the clot removal element 260 to pass through the clot removal element 260 and back into the blood vessel BV. Although it is generally preferred to keep clot material CM within the clot removal element 260, in some instances it can be beneficial to allow the clot removal element 260 to release clot material CM to, e.g., inhibit or prevent damage to the clot removal element 260 that can harm the patient or render the clot removal element 260 inoperable or difficult to operate. Accordingly, as described previously with reference to FIGS. 2A-2D, the relief features 268 can be configured to allow the clot removal element 260 to release clot material CM at forces that are less than the yield strength, the ultimate strength, and/or the fracture strength of the clot removal element 260 and/or one or more of the struts thereof, which is expected to inhibit or even prevent the clot removal element 260 from yielding, breaking, or otherwise failing during a clot treatment procedure.

In some embodiments, at block 1007, the method 1000 includes aspirating clot material from the blood vessel through a lumen of the clot treatment system 100. The aspirated clot material can include all or a portion of the clot material remaining in the blood vessel after the mechanical disruption in block 1006. For example, FIG. 11G shows remaining clot material RCM being aspirated from the blood vessel BV via the second catheter 140. The embolic protection device 136 can remain deployed the aspiration, e.g., to direct the remaining clot material RCM into the second catheter 140 and/or inhibit or even prevent the remaining clot material RCM from embolizing. In these and/or other embodiments, another shaft of the system 100 (e.g., the first catheter 120) can be used to aspirate the remaining clot material RCM and/or other material from the blood vessel BV. In some embodiments, blocks 1006 and 1007 can be repeated until all, or at least a sufficient quantity, of the material in the vasculature is removed, via one or both of mechanical disruption (e.g., block 1006) and aspiration (e.g., block 1007). The sufficient quantity can be at least 50%, 60%, 70%, 80%, 90%, 95%, 99%, etc., of the clot material at a given location in the patient. Although block 1007 is shown as occurring after block 1006 in FIG. 10, in other embodiments block 1007 can occur before block 1006 and/or at a same time as block 1006. After the clot material is sufficiently removed, the method 1000 can include removing the clot treatment system 100 from the patient (block 1008).

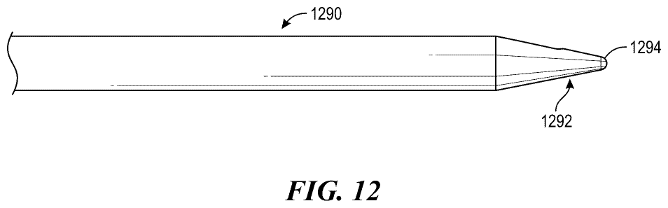

FIG. 12 is a side view of a cleaning tool 1290 configured in accordance with embodiments of the present technology. The cleaning tool 1290 can include an elongate (e.g., cylindrical) body ending at a distal tip portion 1292. The body of the cleaning tool 1290 can be formed from one or more polymers, plastics, metals, compositions, and/or other suitable materials. The distal tip portion 1292 can be tapered radially inwardly toward a distal terminus of the cleaning tool 1294. In some embodiments all, or at least a portion, of the cleaning tool 1290 is coated or other treated to reduce clot formation, improve biocompatibility, reduce friction, and/or otherwise improve handling by a user.

During a clot treatment procedure, the cleaning tool 1290 can be used to clean/remove clot material from within a clot treatment device, such as the clot treatment device 138 of FIG. 1, after the clot treatment device is deployed within a vessel of a patient, used to removed/capture clot material, and then retracted form the vessel. For example, the cleaning tool 1290 can be positioned within the clot treatment device 138 and used to remove clot material caught in one or more of the struts 262 (FIGS. 2A and 2B) and/or otherwise stuck to the clot treatment device 138 etc. For example, inserting the cleaning tool 1290 within the clot treatment device 138 can force any residual clot material with the clot treatment device 138 toward an exterior of the clot treatment device 138, which can make it easier to use the clot treatment device 138 to extract this residual clot material. The cleaning tool 1290 can also be used similar to a guide rail and, accordingly, positioning the cleaning tool 1290 within the clot treatment device 138 can make the clot treatment device easier to handle and/or position. After cleaning the clot treatment device 138 with the cleaning tool 1290, the clot treatment device 138 can be used (e.g., again) to mechanically disrupt the clot material, as described previously with reference to FIG. 11F.

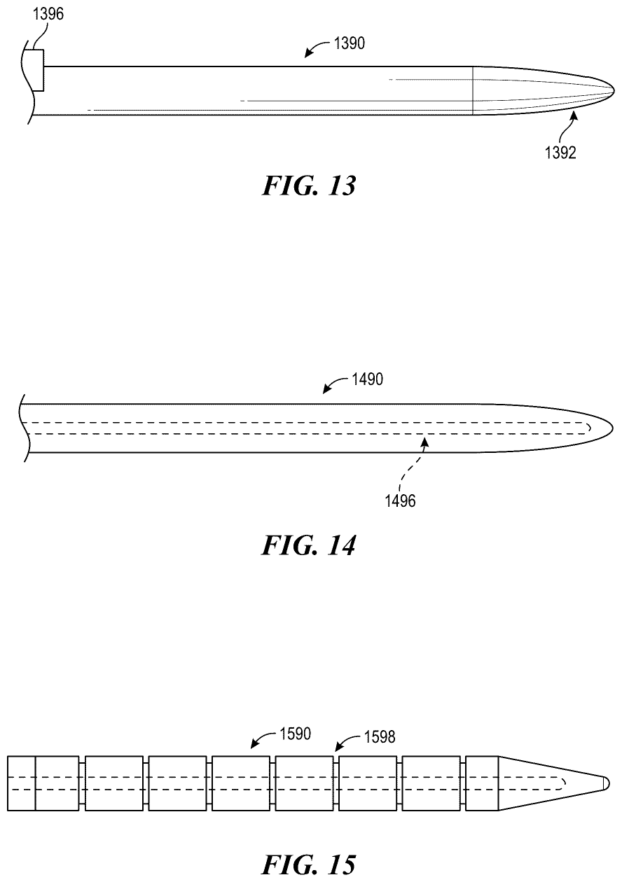

FIG. 13 is a side view of another cleaning tool 1390 configured in accordance with embodiments of the present technology. The cleaning tool 1390 can be generally similar to the cleaning tool 1290 of FIG. 12, but can include a rounded tip 1392 instead of the tapered tip 1292. The cleaning tool 1390 can further include an extension or grip 1396 configured to prevent over-insertion and/or improve ergonomics and handling by the user.

FIG. 14 is a side view of another cleaning tool 1490 configured in accordance with embodiments of the present technology. The cleaning tool 1490 can be generally similar to one or more of the cleaning tools described previously herein, and can further define a hollow interior 1496 configured to increase the flexibility of the cleaning tool 1490 and, accordingly, improve ergonomics and handling by the user.

FIG. 15 is a side view of another cleaning tool 1590 configured in accordance with embodiments of the present technology. The cleaning tool 1590 can be generally similar to one or more of the cleaning tools described previously herein, and can further define one or more annular slots 1598 in an outer surface of the cleaning tool 1590. The annular slots 1598 can be configured to increase the flexibility of the cleaning tool 1490 and/or capture additional residual clot material and, accordingly, improve ergonomics and overall handling by the user.

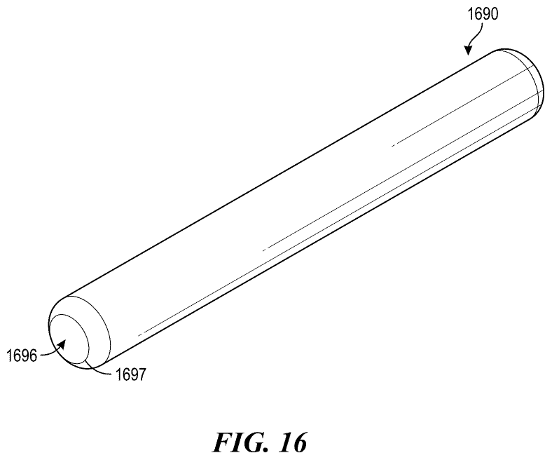

FIG. 16 is a side view of another cleaning tool 1690 configured in accordance with embodiments of the present technology. The cleaning tool 1690 can be generally similar to one or more of the cleaning tools described previously herein, and can further define a hollow interior channel 1696 extending through all, or at least a portion, of the cleaning tool 1690. A proximal end of the cleaning tool 1690 can include a coring tip 1697 configured to remove/core clot material stuck to one or more struts (e.g., one or more of the struts 262 of FIG. 2A) of the clot removal device (e.g., the clot removal element 260 of FIG. 2A).

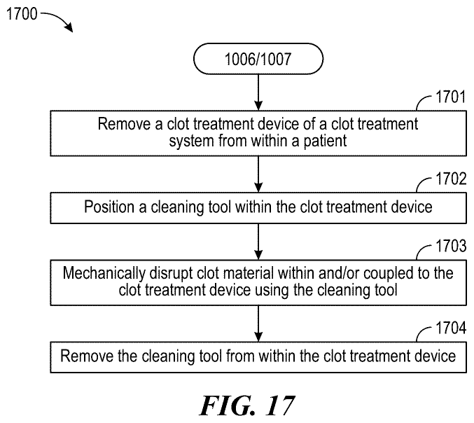

FIG. 17 is a flow diagram of a process or method 1700 for removing clot material from a clot treatment device using a cleaning tool in accordance with embodiments of the present technology. Although some features of the method 1700 are described in the context of the cleaning tool 1290 shown in FIG. 12 for illustration, one skilled in the art will readily understand that the method 1700 can be carried out using other suitable systems and/or devices described herein, including any of the other cleaning tools described herein.

At block 1701, the method 1700 can include removing a clot treatment device of a clot treatment system from within a patient. In at least some embodiments, for example, block 1701 includes removing the clot treatment device 138 (FIG. 2A) from within the blood vessel BV (FIG. 11A) of the patient. In some embodiments, block 1701 can follow block 1006 and/or block 1007 of the method 1000 (FIG. 10). For example, the clot treatment device 138 can be removed from the patient after the clot treatment device 138 is used to mechanically disrupt clot material within the patient (block 1006) and/or after clot material is aspirated using the clot treatment system (block 1007).

At block 1702, the method 1700 can include positioning a cleaning tool of a clot treatment system into a clot treatment device of the clot treatment system. In at least some embodiments, for example, block 1702 includes positioning/inserting the cleaning tool 1290 (FIG. 12) within the clot treatment device 138 (FIG. 2A).

At block 1703, the method 1700 can include mechanically disrupting clot material within and/or coupled to the clot treatment device using the cleaning tool. In at least some embodiments, for example, block 1703 includes advancing or retracting the cleaning tool 1290 relative to the clot treatment device 138 to mechanically disrupt clot material contained within the clot treatment device 138 and/or coupled to one or more of the struts 262 (FIG. 2A) of the clot treatment device 138 after, e.g., the clot treatment device 138 is used to mechanically disrupt clot material within a blood vessel (e.g., block 1006 of FIG. 10). Additionally, or alternatively, positioning the cleaning tool within the clot treatment device can force the clot material to an exterior of the clot treatment device where it can be wiped away via a cloth, picked away via tweezers and/or other instruments, and/or the like. That is, the cleaning tool can cause the clot material to extrude from the cells 264 where it can be more easily removed.

At block 1704, the method 1700 can include removing the cleaning tool from within the clot treatment device. In at least some embodiments, for example, after sufficiently disrupting the clot material within and/or coupled to the clot treatment device 138, the user can remove the cleaning tool 1290 from within the clot treatment device 138. After block 1704, the method 1700 can end, and the clot treatment device 138 can be used again (e.g., in another pass) by, e.g., returning to block 1004 of the method 1000 of FIG. 10.

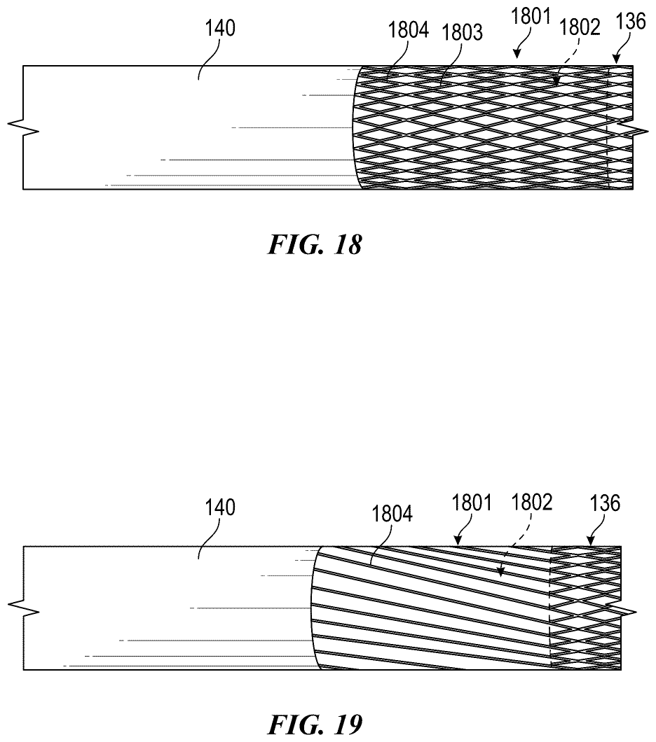

FIGS. 18 and 19 are side views of a proximal or coupling portion 1801 of the embolic protection device 136 (e.g., the funnel 144) of FIG. 1 in accordance with embodiments of the present technology. Referring to FIGS. 18 and 19, the coupling portion 1801 can be coupled to the second catheter 140. For example, in the illustrated embodiment, the coupling portion 1801 extends over and is coupled to an exterior surface portion 1802 at a distal end of the second catheter 140. In other embodiments, the coupling portion 1801 can be coupled to a distal terminus of the second catheter 140 to form, e.g., a butt joint (not shown). Generally, attaching the coupling portion 1801 to the distal terminus of the second catheter 140 via, e.g., a butt joint is expected to reduce the delivery profile (e.g., cross-sectional dimension) of the embolic protection device 136, but this attachment can be weaker/less resilient than attaching the coupling portion 1801 to the exterior surface portion 1802. In contrast, attaching the coupling portion 1801 to the exterior surface portion 1802 of the second catheter 140 is expected to provide a stronger/more resilient attachment but can also increase the delivery profile of the embolic protection device 136. For example, the embolic protection device 136 can include one or more first wires 1803 (FIG. 18 only) that extend along and around a longitudinal axis of the second catheter in a first (e.g., clockwise) direction and/or one or more second wires 1804 that extend longitudinally and radially around the longitudinal axis in a second (e.g., counterclockwise) direction opposite the first direction. Individual ones of the first and second wires 1803, 1804 can overlap/crossover one another in a braided pattern, e.g., on top of the exterior surface portion 1802 of the second catheter 140 to thereby define a delivery profile (e.g., a maximum outer diameter) of the embolic protection device 136. To reduce the delivery profile of the embolic protection device 136 and, by extension, reduce the size of the catheter/sheath used to deliver the embolic protection device 136, all or a subset of the one or more first wires 1803 can be omitted from the coupling portion 1801, as shown in FIG. 19. Omitting all or a subset of the one or more first wires 1803 can reduce, or eliminate entirely, the overlap between the first and second wires 1803, 1804, thereby reducing the delivery profile of the embolic protection device 136 during delivery. In other embodiments, the coupling portion 1801 can include the one or more first wires 1803 and all or a subset of the one or more second wires 1804 can be omitted from the coupling portion 1801. In these and/or other embodiments, the one or more first wires 1803 and/or the one or more second wires 1804 in the coupling portion 1801 can be straightened to extend in a direction at least generally parallel to a longitudinal axis of the second catheter 140 without, or substantially without, extending around the longitudinal axis, thereby reducing, or eliminating entirely, the overlap between the first and second wires 1803, 1804 to thus reduce the delivery profile of the embolic protection device 136 during delivery.

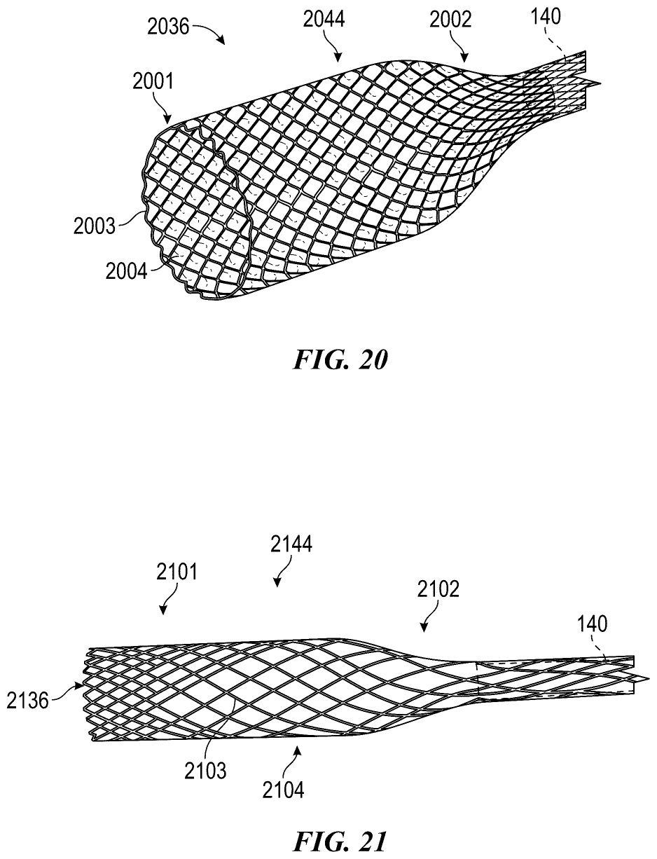

FIG. 20 is a perspective view of an embolic protection device 2036 configured in accordance with additional embodiments of the present technology. In the illustrated embodiment, the embolic protection device 2036 includes a funnel 2044 having a first or distal end portion 2001 and a second or proximal end portion 2002 opposite the distal end portion 2001. The proximal end portion 2002 can taper radially inwardly toward and/or be configured to couple to the second catheter 140 as, e.g., described in detail above with reference to FIGS. 18 and/or 19. The funnel 2044 can include a braided or laser-cut structure 2003 at least partially encapsulated within a coating 2004. The coating 2004 can be configured to inhibit or even prevent fluid flow across the structure 2003 of the funnel 2044. In some embodiments, a thickness of the coating 2004 can vary along the length of the funnel 2044. In the illustrated embodiment, for example, the coating 2004 has a first thickness at the distal end portion 2001 and, at the proximal end portion 2002, has a second thickness greater than the first thickness. In use, the proximal end portion 2002 of the funnel 2044 can experience forces (e.g., radial forces) of greater magnitude than the distal end portion 2001 due to, e.g., the radially inward taper along the proximal end portion 2002 toward the second catheter 140. Increasing the thickness of the coating 2004 at the proximal end portion 2002 can improve the durability and/or strength of the proximal end portion 2002 without, or substantially without, impacting the deliverability of the embolic protection device 2036. For example, because the proximal end portion 2002 has a smaller outer diameter than the distal end portion 2001 when the embolic protection device 2036 is deployed, the proximal end portion 2002 is expected to undergo less of a change when the embolic protection device 2036 is transitioned to the collapsed delivery state and, accordingly, the increased thickness of the coating 2004 at the proximal end portion 2002 is not expected to impact, or substantially impact, the deliverability of the embolic protection device 2036. The decreased thickness of the coating 2004 at the distal end portion 2001 can increase the compliance/flexibility of the distal end portion 2001 and thereby, e.g., optimize the ability of the embolic protection device 2036 to seal the vessel and/or improve other characteristics such as deliverability and/or unsheathing performance. In some embodiments, the coating 2004 can be entirely omitted from the distal end portion 2001 of the funnel 2044. In some embodiments, one or more laser-drilled holes and/or cutouts can be formed through the coating 2004 to, e.g., allow some fluid flow across the structure 2003 and/or otherwise inhibit or even prevent a complete occlusion of the blood vessel when the embolic protection device 2036 is deployed.

FIG. 21 is a side view of an embolic protection device 2136 configured in accordance with additional embodiments of the present technology. In the illustrated embodiment, the embolic protection device 2136 includes a funnel 2144 having a first or distal end portion 2101 and a second of proximal end portion 2102 opposite the distal end portion 2101. The proximal end portion 2102 can taper radially inwardly toward and/or be configured to couple to the second catheter 140 as, e.g., described in detail above with reference to FIGS. 18 and/or 19. The funnel 2144 can include a braided or laser-cut structure comprising a plurality of struts 2103. A density of the struts 2003 can vary along the length of the funnel 2144. In the illustrated embodiment, for example, the funnel 2144 has a first strut density at the distal end portion 2101 and, at the proximal end portion 2102, a second strut density less than (e.g., half of) the first strut density. Increased strut density at the distal end portion 2101 of the funnel 2144 can reduce, or even prevent, unwanted interactions with clot treatment devices (e.g., snagging, catching, etc., on the funnel 2144 when a clot treatment device is withdrawn proximally toward and/or into the funnel 2144). In some embodiments, the funnel 2144 has a first strut density at the distal end portion 2101 and, at the proximal end portion 2102, a second strut density greater than (e.g., double) the first strut density. Increased strut density at the proximal end portion 2102 of the funnel 2144 can increase the durability of the funnel 214 (at least generally similarly to the increased coating thickness described previously with reference to FIG. 20), increase the radially-outward force applied by the funnel 2144 to, e.g., a blood vessel when the funnel 2144 is deployed (which is expected to improve the seal formed between the funnel 2144 and the blood vessel), and/or otherwise improve the ability of the embolic protection device 2136 to inhibit or even prevent blood flow past, through, and/or around the embolic protection device 2136. In some embodiments, the funnel 2144 further includes an intermediate portion 2104 positioned between the distal and proximal end portions 2101, 2101 and the intermediate portion 2104 has a third strut density between the first and second strut densities. In some embodiments, the funnel 2144 can be coated, including with any of the coatings described previously herein.

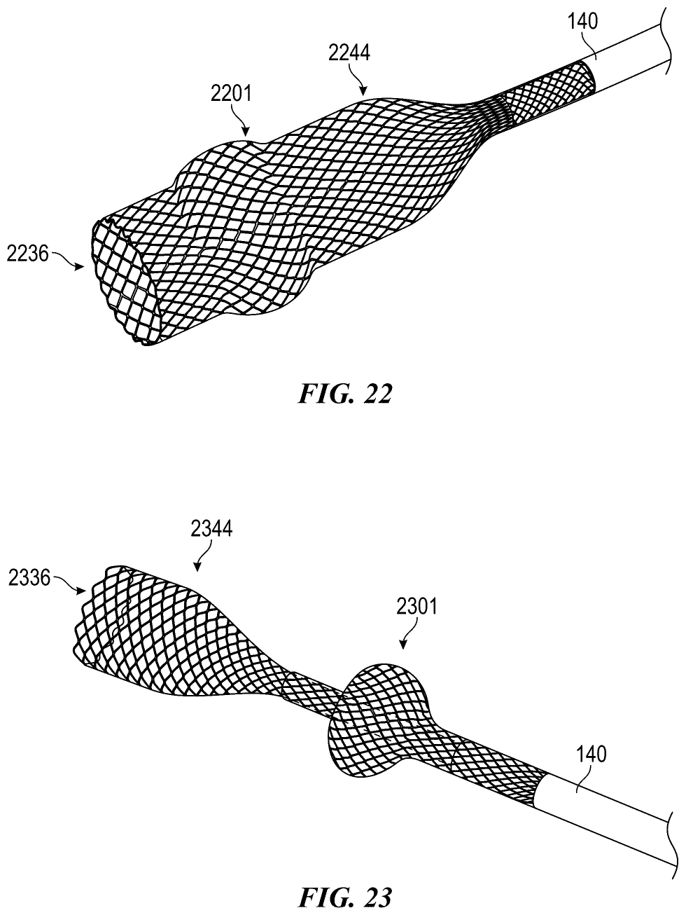

FIG. 22 is a perspective view of an embolic protection device 2236 configured in accordance with additional embodiments of the present technology. In the illustrated embodiment, the embolic protection device 2236 includes a funnel 2244 having a widened portion 2201 (which can also be referred to as a “bump feature,” a “bulb,” a “spherical portion,” and/or the like). In the illustrated embodiment, the widened portion 2201 is located generally medially along the length of the funnel 2244 and extends circumferentially around a longitudinal axis of the embolic protection device 2236. When the funnel 2244 is deployed (within, e.g., a blood vessel), the widened portion 2201 can extend radially outwardly beyond one or more other portions of the funnel 2244 to, e.g., define a maximum radial dimension or extent of the funnel 2244. The widened portion 2201 can improve the ability of the funnel 2244 to inhibit or prevent fluid flow through a blood vessel. For example, when the funnel 2244 is deployed within a blood vessel, the widened portion 2201 can be configured to contact the blood vessel and bend/deform upon contact to, e.g., form an improved substantially fluid-impermeable seal therewith.

FIG. 23 is a perspective view of an embolic protection device 2336 configured in accordance with additional embodiments of the present technology. In the illustrated embodiment, the embolic protection device 2336 includes a funnel 2344 and a bump feature 2301. The bump feature 2301 can be at least generally similar in structure and/or function to the widened portion 2201 described with reference to FIG. 22. However, whereas the widened portion 2201 was part of the funnel 2244, the bump feature 2301 is positioned proximally from the funnel 2344 and, e.g., deployable separately from the funnel 2344. The bump feature 2301 can comprise a same or different construction as the funnel 2344. In the illustrated embodiment, for example, both the bump feature 2301 and the funnel 2344 are braided or laser-cut structures. In other embodiments, the funnel 2344 can be a braided or laser-cut structure, and the bump feature 2301 can include a balloon or other expandable/inflatable element different than the funnel 2344.

Accordingly, described herein are clot treatment systems with clot treatment devices, and associated devices and methods. In some embodiments, a clot treatment system includes an embolic protection device and a clot treatment device. The clot treatment device can include one or more mouths and one or more relief features. The one or more mouths can be configured to mechanically engage clot material within a patient's blood vessel to core or capture at least a portion of the clot material. The one or more relief features can be positioned distal to one or more of the mouths and configured to (i) retain the captured clot material within the clot treatment device and (ii) release the captured clot material when a force on the one or more relief features exceeds a threshold. In at least some embodiments, for example, the relief features can include struts of the clot treatment device that are angled inwardly toward a central axis of the clot treatment device and, when the force on the relief features exceeds the threshold, the relief features can bend or flex outwardly away from the central axis.

During a clot removal procedure, the clot treatment device and the embolic protection device can, while radially constrained within a delivery catheter, be inserted together into a blood vessel of a patient, including the clot material to be treated. The embolic protection device can be deployed at least partially proximal to the clot material and the clot treatment device can be deployed at least partially distal to the clot material. The clot treatment device can be used to mechanically engage and disrupt the clot material by, for example, retracting the one or more mouths proximally through the clot material and into the embolic protection device and/or the first shaft. The relief features can retain the captured clot material within the clot treatment device. If the force on the relief features exceeds the threshold, the relief features can release the captured clot material to reduce the overall force on the clot treatment device to, e.g., prevent the clot treatment device from yielding, breaking, or otherwise failing. The embolic protection device can be positioned to capture all or a portion of the clot material that embolizes or otherwise breaks off during mechanical engagement of the clot treatment device with the clot material and/or to direct clot material into the first shaft.

### III. Selected Embodiments of Mechanical Thrombectomy Assemblies Including Balloon Catheters

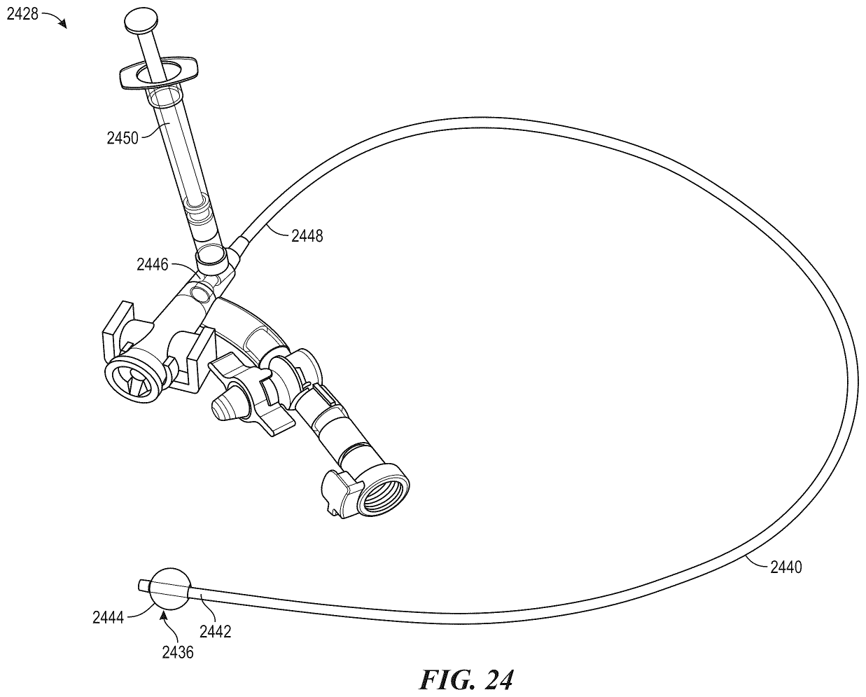

FIG. 24 is a perspective view of an embolic protection catheter assembly 2428 (“assembly 2428”) configured in accordance with embodiments of the present technology. The assembly 2428 can include some features that are at least generally similar in structure and function, or identical in structure and function, to any of the embolic protection catheter assemblies described in detail above with reference to FIGS. 1-23, such as the embolic protection catheter assembly 128 of FIG. 1, and can operate in a generally similar or identical manner to any of those embolic protection catheter assemblies within, for example, the system 100 of FIG. 1. In the illustrated embodiment, the assembly 2428 includes a catheter 2440 coupled to an embolic protection device 2436. The embolic protection device 2436 includes a balloon 2444 at a distal end 2442 of the catheter 2440. The assembly 2428 further includes an inflation port 2446 at a proximal end 2448 of the catheter 2440. The inflation port 2446 can be coupled to a syringe 2450 or other pressure source, and the syringe 2450 can be actuated (e.g., in a first direction) to drive fluid through the inflation port 2446 into the balloon 2444 to inflate the balloon 2444, and can be actuated (e.g., in a second direction) to aspirate fluid through the inflation port 2446 from the balloon 2444 to deflate the balloon 2444. The catheter 2440 can include one or more channels/lumens extending between the balloon 2444 and the inflation port 2446 to route the fluid between the inflation port 2446 and the balloon 2444.

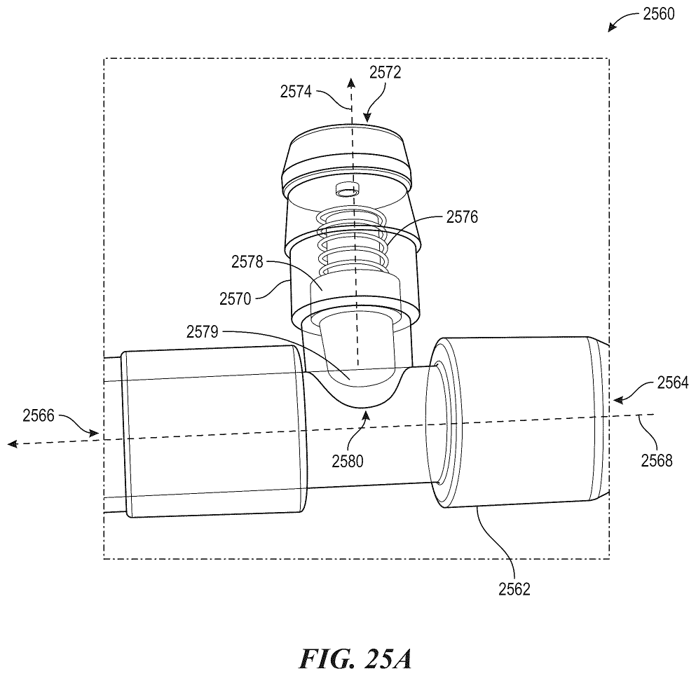

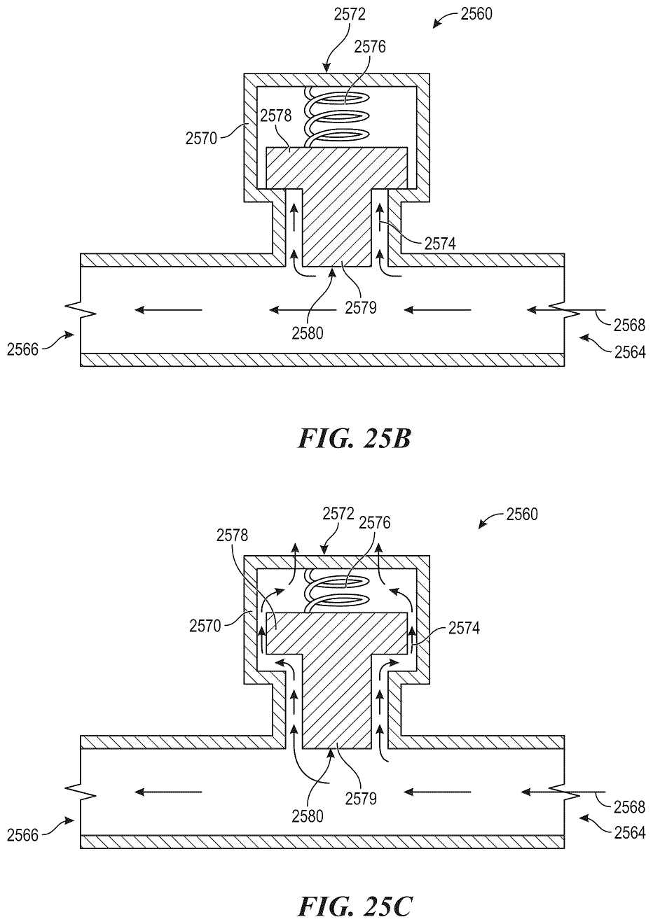

The balloon 2444 can be inflated within a blood vessel to restrict or even halt (e.g., block) blood flow within the vessel to provide embolic protection during a thrombectomy procedure, as described in detail above. In some embodiments, the fluid comprises a contrast media to allow a user to visualize (e.g., fluoroscopically) the balloon 2444 in real time. To reduce, or even eliminate, damage to the balloon 2444, an inflation control device (e.g., inflation control device 2560 (FIG. 25)) can be positioned along a fluid path between the syringe 2450 and the balloon 2444.

FIG. 25A is a side view of an inflation control device 2560 in accordance with embodiments of the present technology. FIGS. 25B and 25C are cross-sectional views of the inflation control device 2560 of FIG. 25A in a closed position and an open position, respectively, in accordance with embodiments of the present technology. Referring to FIGS. 25A-25C together, the inflation control device 2560 can be a pressure relief valve, which prevents overinflation of the balloon 2444 (FIG. 24) caused by injecting the contrast media or other fluid too quickly into the balloon 2444. The inflation control device 2560 includes a conduit 2562 that defines an inlet 2564 and an outlet 2566 with a fluid path 2568 extending between the inlet 2564 and the outlet 2566. A fluid (e.g., the contrast media) can flow from the inlet 2564 to the outlet 2566 along the fluid path 2568. For example, the fluid can flow from the syringe 2450 (FIG. 24) through the inlet 2564, along the fluid path 2568, and out of the outlet 2566 of the inflation control device 2560 toward the balloon 2444.

In the illustrated embodiment, the inflation control device 2560 further comprises a pressure relief conduit 2570 that defines a relief outlet 2572 and a relief fluid path 2574 extending through the pressure relief conduit 2570 and in fluid communication with the fluid path 2568. Referring to FIG. 25A, the pressure relief conduit 2570 can extend perpendicularly to the conduit 2562. The relief outlet 2572 can be fluidly connected to a collection device (not shown), such as a bag or a syringe, to capture and/or divert fluid exiting the relief outlet 2572. A biasing member 2576 and a stopper 2578 can be in fluid communication with the relief fluid path 2574 and configured to restrict (e.g., eliminate) flow out of the relief outlet 2572 until a predetermined condition is satisfied. A free end 2579 (“stopper end 2579”) of the stopper 2578 extends into an opening 2580 positioned in a wall of the conduit 2562. The opening 2580 is located between the inlet 2564 and the outlet 2566, and the free end 2579 of the stopper 2578 can inhibit the flow of fluid through the opening 2580 and into the pressure relief conduit 2570. The biasing member 2576 can be made integral with the stopper 2578 and biases the stopper end 2579 toward (e.g., through) the opening 2580. The opposite end of the biasing member 2576 is in contact with a feature (not shown) of the pressure relief conduit 2570 in proximity to the relief outlet 2572. By connecting the biasing member 2576 between the stopper 2578 and the feature (not shown), the biasing member 2576 is maintained within the pressure relief conduit 2570, and in turn, maintains the position of the stopper 2578 within the pressure relief conduit 2570 and the opening 2580.

In some embodiments, the predetermined condition can include a pressure increase above a threshold value, which can overcome a biasing force (e.g., spring rate) of the biasing member 2576 and move the stopper 2578 toward the relief outlet 2572, causing the stopper end 2579 to move away from the opening 2580. The stopper 2578 is thereby moved from a closed position to an open position, thereby allowing fluid to flow from the inlet 2564 to the relief outlet 2572. The pressure at which the biasing member 2576 is overcome can be referred to as the “cracking pressure.” Referring to FIG. 25B, the stopper 2578 of the inflation control device 2560 is in the closed position because the cracking pressure of the fluid does not exceed the biasing force of the biasing member 2576. Thus, the fluid is directed from the inlet 2564 to the outlet 2566. More specifically, fluid does not enter the pressure relief conduit 2570. Referring to FIG. 25C, the stopper 2578 of the inflation control device 2560 is in the open position because the cracking pressure of the fluid exceeds the biasing force of the biasing member 2576. Thus, the fluid is directed from the inlet 2564 to the relief outlet 2572 through the pressure relief conduit 2570. As illustrated, the stopper end 2579 of the stopper 2578 moves away from the opening 2580 to allow the fluid to flow from the inlet 2564 to the relief outlet 2572. At the same time, fluid can flow from the inlet 2564 to the outlet 2566.

In some embodiments, pressure above 10 pounds per square inch (psi) can overcome the biasing force of the biasing member 2576 and move the stopper 2578 from the closed position (FIG. 25B) to the open position. (FIG. 25C) In other embodiments, the pressure can be customized by adjusting the biasing force of the biasing member 2576. The cracking pressure can be varied, selected, and/or optimized based in part on the type of balloon 2444 (FIG. 24) selected. During operation of the inflation control device 2560, when the inlet 2564 pressure exceeds the cracking pressure of the inflation control device 2560, the stopper 2578 will displace toward the relief outlet 2572 to allow the fluid, such as the contrast media, to exit through the relief outlet 2572 to keep the assembly 2428 pressure below the cracking pressure and thereby inhibiting, or even preventing, damage to the balloon 2444 that may be caused by over inflation. Generally, the cracking pressure can be set below a pressure that can cause damage to the balloon 2444 (e.g., the rated burst pressure). Thus, if a user injects a fluid too quickly, the cracking pressure will not allow the assembly 2428 pressure to rise to the rated burst pressure of the balloon 2444.

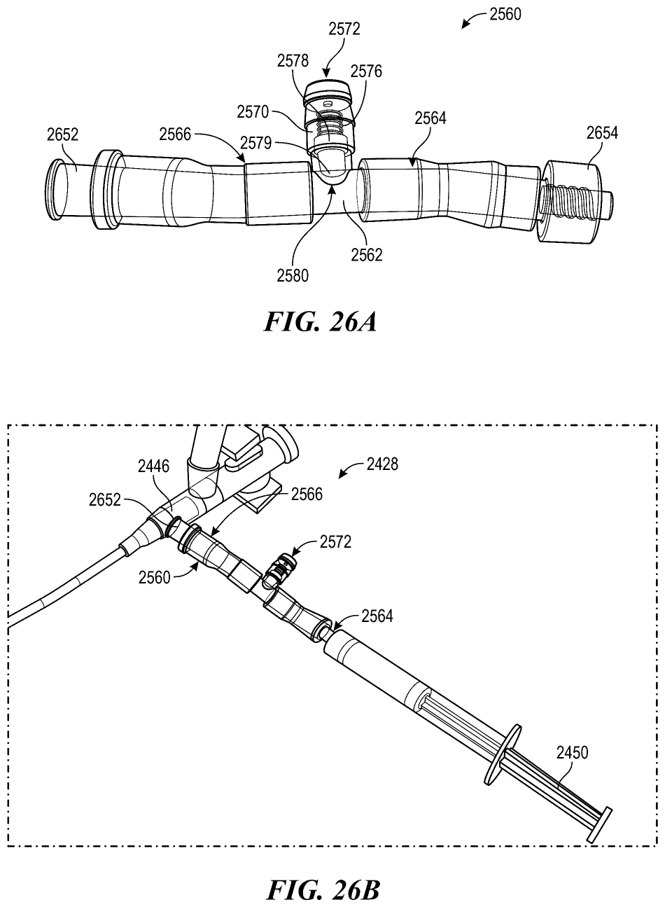

FIG. 26A is a side view of the inflation control device 2560 of FIG. 25A assembled with additional adapters in accordance with embodiments of the present technology. FIG. 26B is a perspective view of the inflation control device 2560 of FIG. 25A fluidly connected between the syringe 2450 and the inflation port 2446 of the assembly 2428 of FIG. 24 in accordance with embodiments of the present technology. Referring to FIGS. 26A and 26B together, the inflation control device 2560 is positioned between the inflation port 2446 and the syringe 2450. More specifically, the inlet 2564 of the inflation control device 2560 is positioned in fluid communication with the syringe 2450, and the outlet 2566 of the inflation control device 2560 is positioned in fluid communication with the inflation port 2446. In some embodiments, the inlet 2564 of the inflation control device 2560 can be in direct or indirect contact with the syringe 2450. In other embodiments, the outlet 2566 of the inflation control device 2560 can be in direct or indirect contact with the inflation port 2446. In further embodiments, the inlet 2564 of the inflation control device 2560 can be in direct or indirect contact with a syringe adapter 2652 (FIG. 26A). The syringe adapter 2652 can be in fluid communication with the inflation control device 2560 and the syringe 2450. In some embodiments, the outlet 2566 of the inflation control device 2560 can be in direct or indirect contact with an inflation port adapter 2654 (FIG. 26A). The inflation port adapter 2654 can be in fluid communication with the inflation control device 2560 and the inflation port 2446. The inflation control device 2560 can be removably coupled to the inflation port 2446 and the syringe 2450.

Referring to FIGS. 26A and 26B, the syringe adapter 2652 can be in fluid communication with the inflation control device 2560 and the syringe 2450. In some embodiments, the syringe 2450 can be removably coupled to the inflation control device 2560. Further, the inflation control device 2560 can be mounted with respect to the syringe 2450. For example, the inflation control device 2560 can be integrated with the syringe 2450 such that the inflation control device 2560 and the syringe 2450 are fluidly connected to the inflation port 2446. For example, the outlet 2566 of the inflation control device 2560 can be in direct or indirect connection with the inflation port 2446. In some embodiments, the outlet 2566 of the inflation control device 2560 can be in direct or indirect connection with the inflation port adapter 2654 (FIG. 26A). In some embodiments, the inflation port adapter 2654 can be in fluid communication with the inflation control device 2560 and the inflation port 2446. In some embodiments, the inflation control device 2560 and the syringe 2450 can be removably coupled to the inflation port 2446.

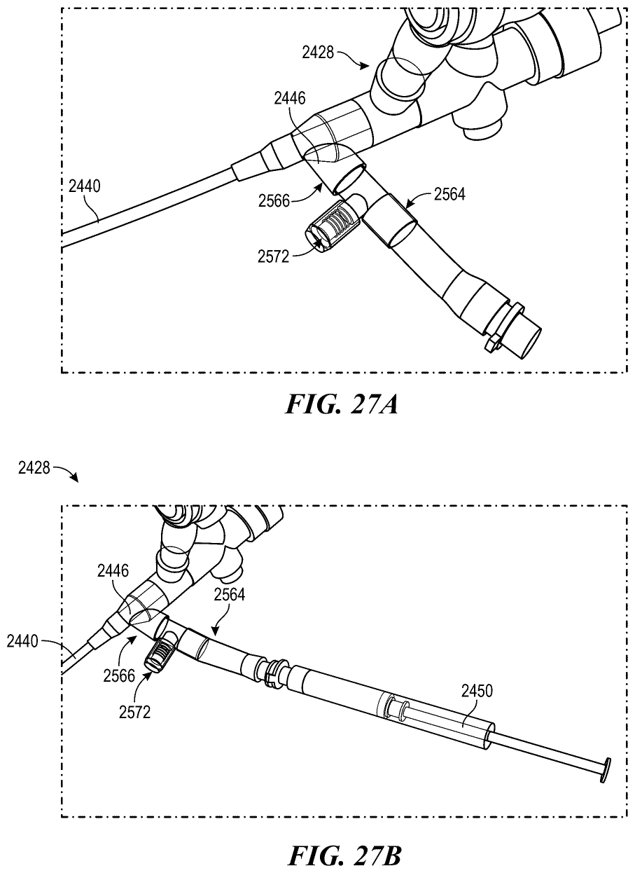

FIG. 27A is a perspective view of the inflation control device 2560 of FIG. 25A mounted to the inflation port 2446 of the assembly 2428 of FIG. 24 in accordance with embodiments of the present technology. FIG. 27B is a perspective view of the inflation control device 2560 of FIG. 27A with the syringe 2450 fluidly connected to the inlet 2564 of the inflation control device 2560 in accordance with embodiments of the present technology. Referring to FIGS. 27A and 27B together, the inflation control device 2560 is integrated with the inflation port 2446 of the catheter 2440 such that the inflation control device 2560 extends outwardly from the catheter 2440. Thus, the outlet 2566 of the inflation control device 2560 is fixedly coupled to the inflation port 2446 of the catheter 2440 such that the inflation control device 2560 is integrated within the catheter 2440.

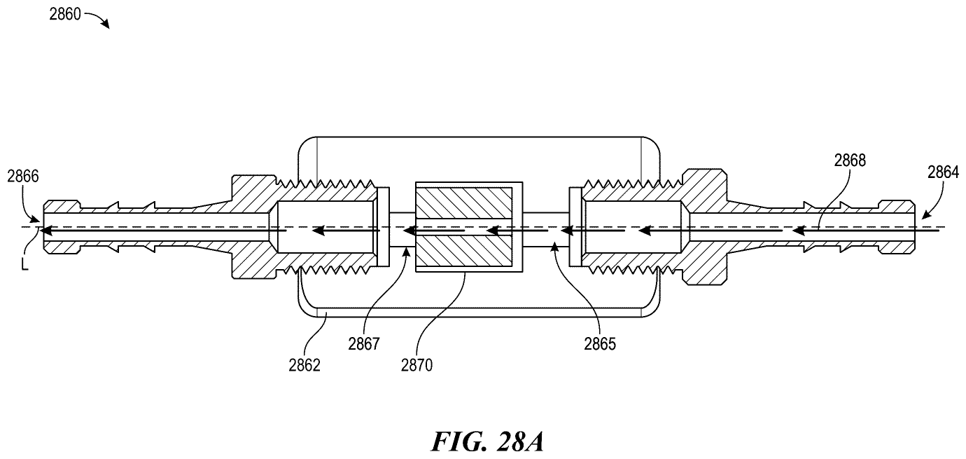

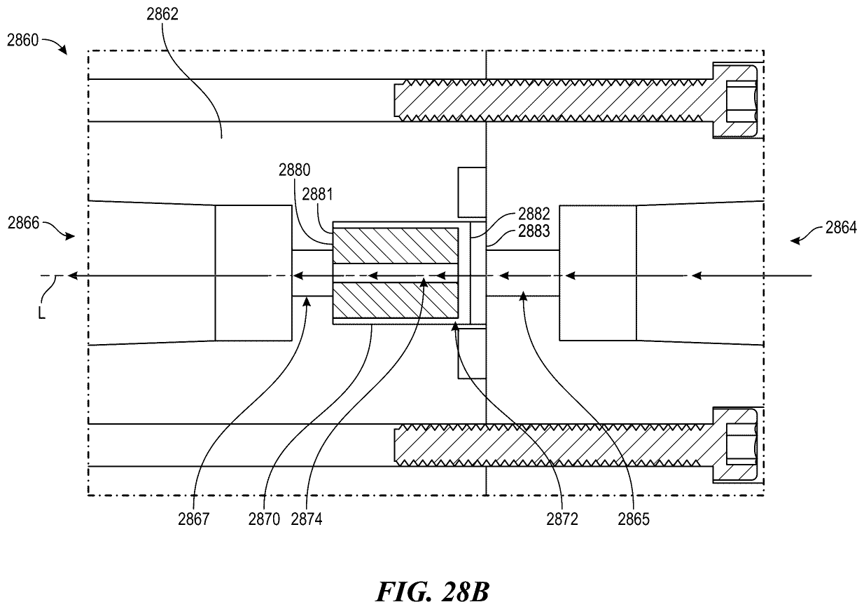

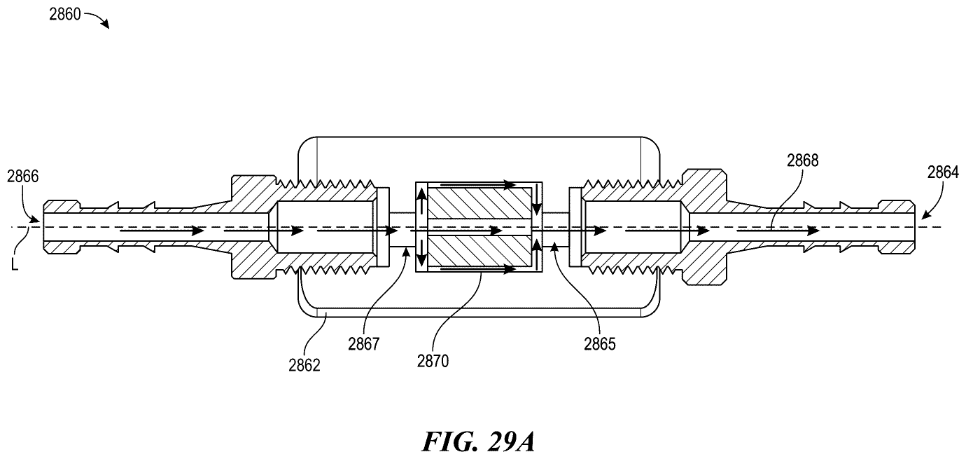

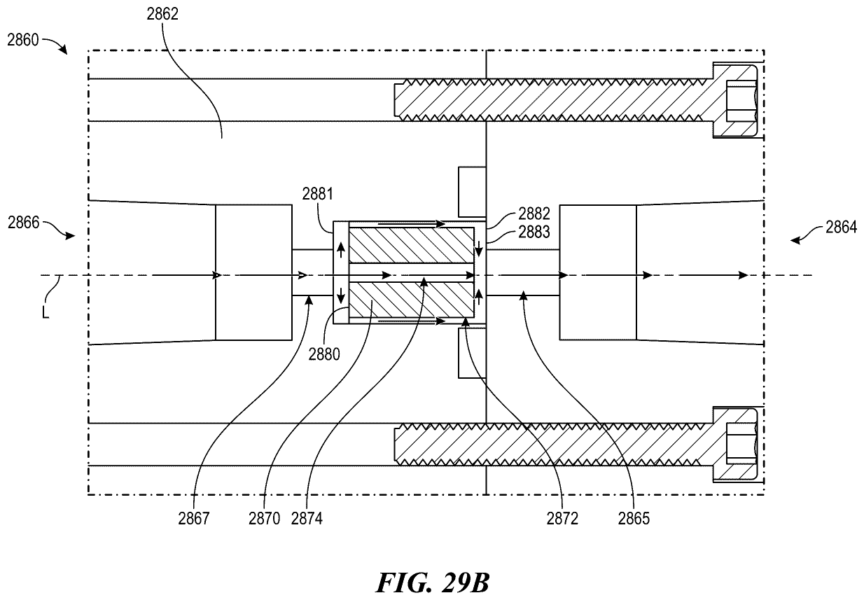

FIG. 28A is a side view of an inflation control device 2860 in a first position in accordance with additional embodiments of the present technology. FIG. 29A is a side view of the inflation control device 2860 of FIG. 28A in a second position in accordance with embodiments of the present technology. Referring to FIGS. 28A and 29A together, the inflation control device 2860 can include some features that are at least generally similar in structure and function, or identical in structure and function, to the corresponding features of the inflation control device 2560 described in detail above with reference to FIGS. 25A-27B, and can operate in a generally similar or identical manner to the inflation control device 2560. For example, the inflation control device 2860 includes a housing 2862 that defines an inlet 2864 and an outlet 2866 and a fluid path 2868 that fluidly connects the inlet 2864 and outlet 2866. In some embodiments, the fluid path 2868 can be defined by a single passageway that fluidly connects the inlet 2864 and the outlet 2866. In other embodiments, the fluid path 2868 can vary in diameter between the inlet 2864 and the outlet 2866. Although the inlet 2864 and the outlet 2866 are illustrated as fittings, in some embodiments, alternate connections can be used, such as a Luer lock fitting, a barb fitting, a slip fitting, a quick-connect fitting, an adhesive attachment, and/or the like. In the illustrated embodiment, the inflation control device 2860 includes an inlet fluid channel 2865 and an outlet fluid channel 2867. In some embodiments, the inlet fluid channel 2865 and the outlet fluid channel 2867 can be defined by multiple passageways that fluidly couple the inlet 2864 and the outlet 2866. In some embodiments, a fluid, such as contrast medium, can flow from the inlet 2864 to the outlet 2866 along the fluid path 2868. For example, the fluid can flow from the syringe 2450 (FIG. 24) through the inlet 2864, along the fluid path 2868, and out the outlet 2866 of the inflation control device 2860 toward the balloon 2444 (FIG. 24).

In the illustrated embodiment, the inflation control device 2860 includes a restrictor 2870, which is configured to move between a first position and a second position along a longitudinal axis L of the inflation control device 2860. For example, the restrictor 2870 can be at least partially positioned within the fluid path 2868 and be movable in a direction toward and away from either the inlet 2864 or the outlet 2866 along the axis L. In some embodiments, the restrictor 2870 can be sized and shaped to enable fluid to flow at least partially through and around the restrictor 2870, as described in greater detail below.

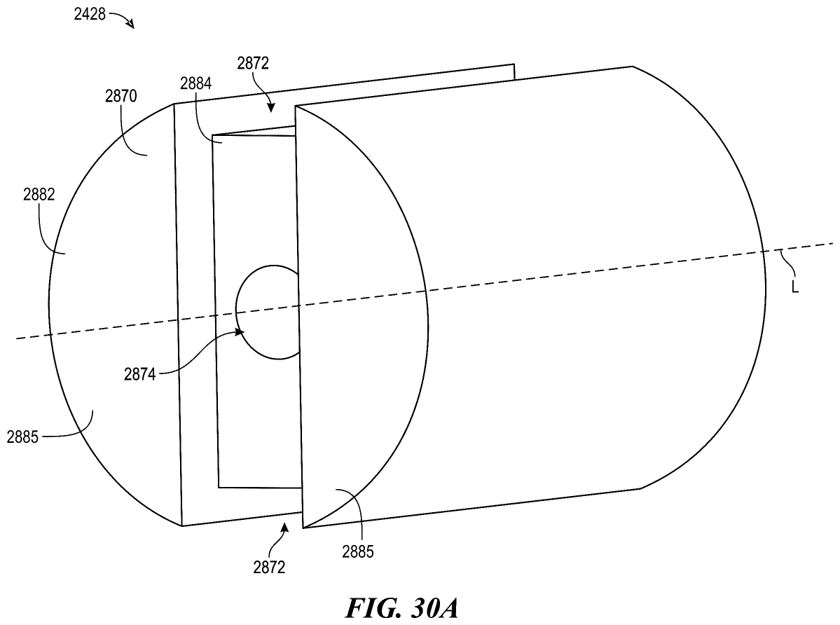

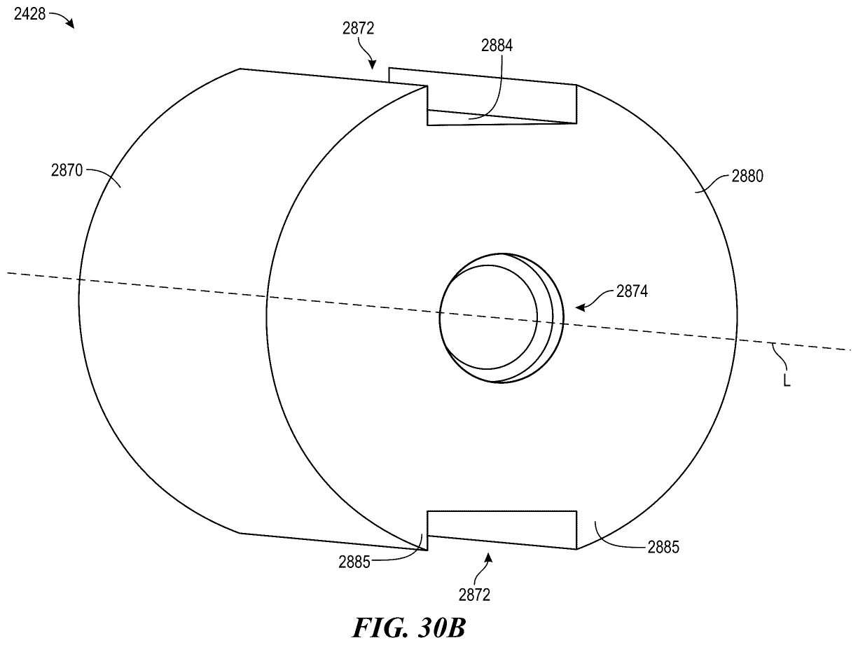

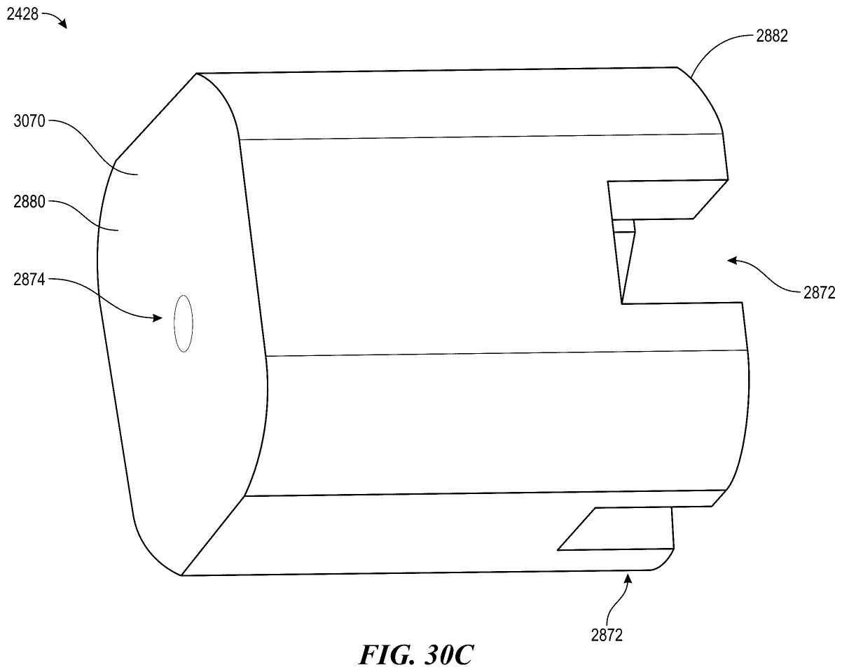

FIG. 28B is an enlarged cross-sectional view of the inflation control device 2860 in the first position of FIG. 28A in accordance with embodiments of the present technology. FIG. 29B is an enlarged cross-sectional view of the inflation control device 2860 in the second position of FIG. 29A in accordance with embodiments of the present technology. Referring to FIGS. 28B and 29B together, the restrictor 2870 defines one or more first fluid channels 2872 (“first fluid channels 2872”; e.g., slots, holes, fluid passage) and a second fluid channel 2874. The one or more first fluid channels 2872 are positioned along the outer surface of the restrictor 2870, as described in greater detail below with reference to FIG. 30. The second fluid channel 2874 can be a circular lumen that extends through the restrictor 2870 along the axis L. In the illustrated embodiment, the restrictor 2870 includes (i) a first (e.g., distal) face 2880 that faces the outlet 2866 and a first (e.g., distal) flange wall 2881 defined within the housing 2862 and (ii) a second (e.g., proximal) face 2882 opposite the first face 2880 and that faces the inlet 2864 and a second (e.g., proximal) flange wall 2883 defined within the housing 2862.

FIGS. 30A and 30B are front and back perspective views, respectively, of the restrictor 2870 of the inflation control device 2860 of FIG. 28A in accordance with embodiments of the present technology. Referring to FIGS. 30A and 30B together, the restrictor 2870 defines the first fluid channels 2872 and the second fluid channel 2874. More specifically, the restrictor 2870 includes a middle portion 2884 connected on opposite sides to two semicircular portions 2885. The second fluid channel 2874 extends through the middle portion 2884, and the semicircular portions 2885 and opposite sides of the middle portion 2884 together define the first fluid channels 2872. Referring to FIG. 30B, in the illustrated embodiment the middle portion 2884 terminates away from the first face 2880 of the restrictor 2870 such that the first fluid channels 2872 are in fluid communication with the second fluid channel 2874 at/proximate the first face 2880. In contrast, referring to FIG. 30B, the middle portion 2884 can terminate at the second face 2882 with the semicircular portions 2885. In other embodiments, the restrictor 2870 can include more or fewer of the first fluid channels 2872, depending on the desired flow, and/or the restrictor 2870 and portions thereof can have different shapes.

FIG. 30C is a perspective view of a restrictor 3070 of the inflation control device 2860 of FIG. 28A in accordance with additional embodiments of the present technology. In the illustrated embodiment, the restrictor 3070 includes four first fluid channels 2872. Further, the restrictor 3070 is generally circular, and the first fluid channels 2872 are created by removing material from an external surface of the restrictor 3070.

Referring to FIGS. 28A and 28B together, the first position of the restrictor 2870 corresponds to fluid flow from the inlet 2864 to the outlet 2866 (e.g., during inflation of the balloon 2444). As a fluid travels from the inlet 2864 to the outlet 2866 of the inflation control device 2860, the fluid moves the restrictor 2870 toward the outlet 2866 such that the first face 2880 of the restrictor 2870 contacts/abuts the first flange wall 2881 in the first position. In that first position, (i) the contact between the first face 2880 and the first flange wall 2881 blocks the first fluid channels 2872—that is, there is no flow path between the first fluid channels 2872 and the second fluid channel 2874—for fluid to flow through them from the inlet 2864 to the outlet 2866—and (ii) the second fluid channel 2874 is not blocked (e.g., unblocked) such that fluid can flow through the second channel 2874 to the outlet 2866. As such, the fluid travels from the inlet 2864 along the fluid path 2868 only through the second fluid channel 2874 of the restrictor 2870 and out the outlet 2866 and not through the first fluid channels 2872. More specifically, the fluid travels from the inlet 2864 sequentially through (i) the inlet fluid channel 2865, (ii) the second fluid channel 2874, (iii) the outlet fluid channel 2867, and (iv) the outlet 2866.

Referring to FIGS. 29A and 29B together, the second position corresponds to fluid flow from the outlet 2866 to the inlet 2864, opposite the direction of fluid movement in the first position (e.g., during deflation of the balloon 2444). As a fluid travels from the outlet 2866 to the inlet 2864 of the inflation control device 2860, the fluid moves the restrictor 2870 toward the inlet 2864 such that the second face 2882 of the restrictor 2870 contacts/abuts the second flange wall 2883 in the second position. In that second position, (i) the first fluid channels 2872 are not blocked (e.g., fluid can flow through them to the inlet 2864) because fluid can flow between the middle portion 2884 (FIGS. 30A and 30B) of the restrictor 2870 and the second flange wall 2883 into the first fluid channels 2872 because the middle portion 2884 is set back from the second face 2882 and the second flange wall 2883, and (ii) the second fluid channel 2874 is not blocked such that fluid can flow through the second channel 2874 to the inlet 2864. In other embodiments, the second fluid channel 2874 can be blocked in the second position depending on the desired fluid flow. In the illustrated embodiment, the fluid flows from the outlet 2866 along the fluid path 2868 and through the first fluid channels 2872 and the second fluid channel 2874 of the restrictor 2870 and out of the inlet 2864. More specifically, the fluid travels from the outlet 2866 sequentially through (i) the outlet fluid channel 2867, (ii) one of the first fluid channels 2872 or the second fluid channel 2874 of the restrictor 2870, (iii) the inlet fluid channel 2865, and (iv) the inlet 2864.

In some embodiments, the fluid flow can be distributed evenly or can be distributed unevenly between the first fluid channels 2872 and the second fluid channel 2874 of the restrictor 2870. For example, the fluid flow through the first fluid channels 2872 of the restrictor 2870 can be greater than the fluid flow through the second fluid channel 2874 of the restrictor 2870. In other embodiments, the fluid flow through the second fluid channel 2874 of the restrictor 2870 can be greater than the flow through the first fluid channels 2872 of the restrictor 2870.

In the illustrated embodiment, the fluid flow is faster in the first position (e.g., deflation of the balloon 2444) than in the second position (e.g., during inflation of the balloon 2444). In general, during deflation of the balloon 2444, the fluid flow moving from the outlet 2866 to the inlet 2864 will have fewer restrictions compared to the fluid flow moving from the inlet 2864 to the outlet 2866, which can help the balloon 2444 deflate more quickly than it is inflated. In some aspects of the present technology, this can reduce, or even prevent, damage from inflating the balloon 2444 too quickly. For example, when the user inflates the balloon 2444 too much or too quickly, they can recognize this and immediately begin deflating the balloon 2444 at an accelerated rate. This serves to inhibit damage to the balloon 2444 in two ways: (i) the balloon 2444 can quickly deflate when overinflated, and (ii) the reduced flow rate for inflation inhibits overinflation as the inflation is more incremental and overinflation can be identified before it damages the balloon 2444. In some embodiments, the inflation flow rate into the balloon 2444 can be about 0.1 cubic centimeters/second (“cc/sec”) or less.

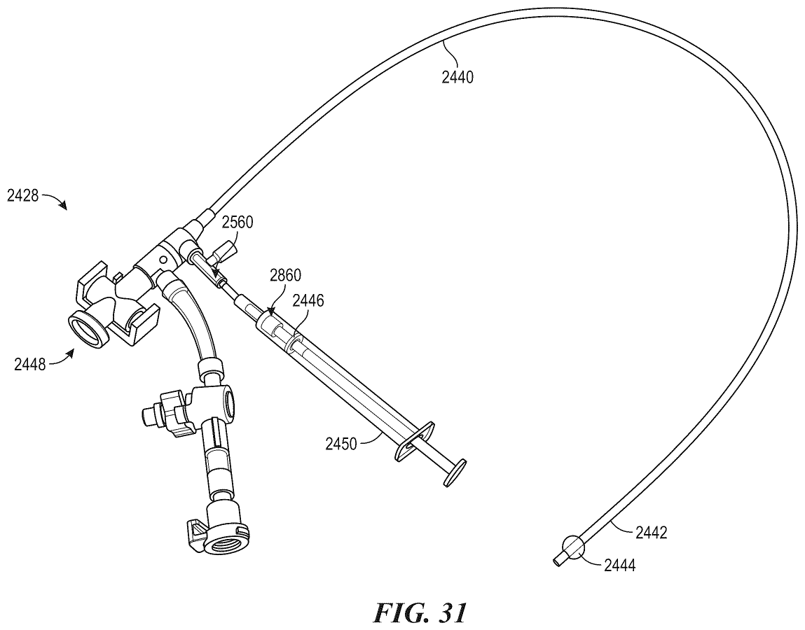

FIG. 31 is a perspective view of the assembly 2428 of FIG. 24 including two inflation control devices configured in accordance with embodiments of the present technology. More specifically, in the illustrated embodiment, the assembly 2428 includes the inflation control device 2560 (FIGS. 25A-27B) and the inflation control device 2860 (FIGS. 28A-30). In the illustrated embodiment, to reduce or even eliminate damage to the balloon 2444, the inflation control device 2560 and the inflation control device 2860 are positioned along a fluid path between the syringe 2450 and the balloon 2444. In some instances, rapidly injecting fluid can create a pressure differential between the inflation control device 2860 and the balloon 2444 such that the inflation control device 2560 exceeds its cracking pressure prior to fluid reaching the balloon 2444. This can produce an under-inflated balloon despite the inflation control device 2560 functioning as intended. Although reducing the flow rate in the catheter 2440 can decrease the likelihood that the balloon 2444 will be damaged, the balloon 2444 needs to be able to deflate quickly, and thus, a reduced flow rate can be a disadvantage. Accordingly, the inflation control device 2860 can be used in tandem with the inflation control device 2560 to quickly deflate the balloon 2444.

In some embodiments, to deflate the balloon 2444, the syringe 2450 is pulled in a direction opposite the direction pressed during inflation, which aspirates the fluid (e.g., contrast media) from the balloon 2444. More specifically, to inflate the balloon 2444, positive pressure can be generated in the syringe 2450 (e.g., via depression of a plunger of the syringe 2450) to force fluid through the inflation control devices 2560, 2860 and toward the balloon 2444, and to deflate the balloon 2444, negative pressure can be generated in the syringe 2450 (e.g., via withdrawal of a plunger of the syringe 2450) to pull fluid through the inflation control devices 2560, 2860 and toward the syringe 2450.

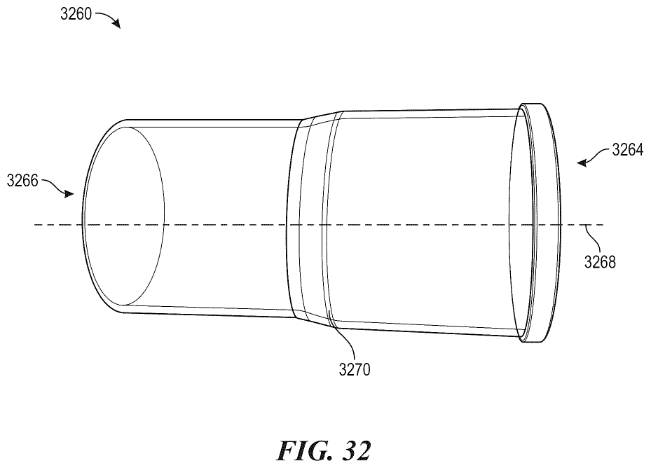

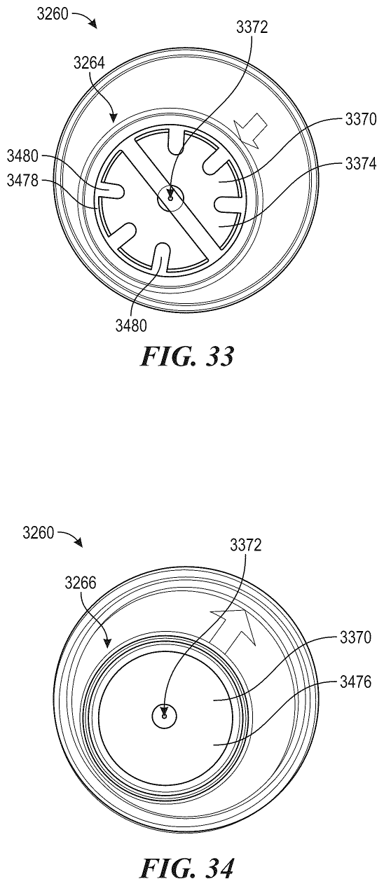

FIG. 32 is a side view of an inflation control device 3260 in accordance with additional embodiments of the present technology. FIG. 33 is a first end view of the inflation control device 3260 of FIG. 32 in accordance with embodiments of the present technology. FIG. 34 is a second end view of the inflation control device 3260 of FIG. 32 in accordance with embodiments of the present technology. Referring to FIGS. 32-34 together, the inflation control device 3260 can include some features that are at least generally similar in structure and function, or identical in structure and function, to the corresponding features of the inflation control devices 2560, 2860 described in detail above with reference to FIGS. 24-30, and can operate in a generally similar or identical manner to the inflation control devices 2560, 2860. In some embodiments, the inflation control device 3260 can be positioned along the fluid path between the syringe 2450 (FIG. 24) and the balloon 2444 (FIG. 24). In the illustrated embodiment, the inflation control device 3260 is a flow restrictor valve that can inhibit overinflation of the balloon 2444, which at times is caused by injecting fluid (e.g., contrast media) too quickly into the balloon 2444. In some embodiments, the assembly 2428 can further include the inflation control device 3260, as described herein.

Referring to FIG. 32, the inflation control device 3260 includes an inlet 3264 and an outlet 3266 with a fluid path 3268 therebetween. The inflation control device 3260 further includes a restrictor 3270 (e.g., a flap) that moves between an open position and a closed position. In some embodiments, the inflation control device 3260 can be configured to at least partially cover the flow path of the inflation port 2446 (FIG. 24).

Referring to FIGS. 33 and 34 together, the restrictor 3270 defines a fluid channel 3372 that extends between a first side 3374 and a second side 3476 of the restrictor 3270. In the illustrated embodiment, the fluid channel 3772 allows fluid to pass between the inlet 3264 and the outlet 3266. The first side 3374 is positioned relative to the inlet 3264, and the second side 3476 is positioned relative to the outlet 3266.

Referring to FIG. 33, the inflation control device 3260 can include a stop feature 3478 (e.g., ledge, lip) that is circumferentially positioned relative to the inner diameter of the inflation control device 3260 on the first side 3374 of the restrictor 3270. In some embodiments, the stop feature 3478 can act as a sealing surface for the restrictor 3270. In the illustrated embodiment, the inflation control device 3260 also includes a plurality of fingers 3480 that extend inwardly from the stop feature 3478 in the direction of the fluid channel 3372. In some aspects of the present technology, the plurality of fingers 3480 can provide support to the restrictor 3270 when in the closed position. In some embodiments, the stop feature 3478 and the plurality of fingers 3480 can be planar relative to each other.

Referring to FIGS. 32-34 together, during operation, the restrictor 3270 opens to allow flow to pass through in one flow direction (e.g., deflation) and pushes closed in another flow direction (e.g., inflation). For example, during inflation, fluid flows through the inlet 3264 and pressure builds on the first side 3374 of the restrictor 3270. The stop feature 3478 and the plurality of fingers 3480 provide support to the restrictor 3270 and inhibit the restrictor 3270 from flexing, causing the fluid to flow primarily through the restricted fluid channel 3372. In contrast, during deflation, when fluid flow contacts the second side 3476 of the restrictor 3270, it causes the restrictor 3270 to flex open and allow unrestricted flow from the outlet 3266 to the inlet 3264. More specifically, the restrictor 3270 flexes open because the restrictor 3270 is not supported by the stop feature 3478 on the second side 3476. Thus, the inflation control device 3260 can limit the amount of flow being delivered to the balloon 2444 during inflation of the balloon 2444 and can freely allow fluid flow when the balloon 2444 is being deflated.

### IV. Additional Examples

Several aspects of the present technology are set forth in the following examples:
1. A clot treatment device for treating clot material within a blood vessel of a patient, the clot treatment device comprising:
a unitary structure including a plurality of interconnected struts, wherein the unitary structure defines a central axis and includes—
   a first region defining a coupling portion configured to couple the unitary structure to a catheter;
   a second region distal of the first region and defining a proximal mouth configured to capture at least a portion of the clot material;
   a third region distal of the second region and defining one or more relief features angled inwardly toward the central axis and configured to (i) retain the captured portion of the clot material between the proximal mouth and the one or more relief features when a force on the one or more first relief features is below a threshold and (ii) rotate away from the central axis to release the captured portion of the clot material when the force equals or exceeds the threshold; and
   a fourth region distal of the second region and defining a distal terminus of the unitary structure.
2. The clot treatment device of example 1 wherein, in the third region, a subset of the struts terminate at junctions to define the one or more relief features.
3. The clot treatment device of example 1 wherein threshold is based at least in part on the inward angle of the one or more relief features.
4. The clot treatment device of example 1 wherein a subset of the struts define the one or more relief features, and wherein the threshold is based at least in part on a thickness of the subset of the struts.
5. The clot treatment device of example 1 wherein the second, third, and fourth region define a clot removal element of the clot treatment device; and wherein the second region further defines a leading edge portion of the clot removal element that has a thickness that varies along its length.
6. The clot treatment device of example 5 wherein the leading edge portion spans a subset of the plurality of interconnected struts, and wherein the thickness of the leading edge portion (i) increases in a proximal-to-distal direction between immediately adjacent ones of the subset of the plurality of interconnected struts and (ii) decreases between immediately adjacent pairs of the plurality of interconnected struts.
7. The clot treatment device of example 5 wherein—
the plurality of interconnected struts include (i) a first proximal strut coupled to the leading edge portion, (ii) a second proximal strut coupled to the leading edge portion distal to the first proximal strut, and (ii) a third proximal strut coupled to the leading edge portion distal to the second proximal strut;
the leading edge portion has (i) a first segment between the first and second proximal struts and (ii) a second segment between the second and third proximal struts;
the first segment has (i) a first thickness at the first proximal strut and (ii) a second thickness greater than the first thickness at the second proximal strut; and
the second segment has (i) a third thickness less than the second thickness at the second proximal strut and (ii) a fourth thickness greater than the third thickness at the third proximal strut.
8. The clot treatment device of example 7 wherein the fourth thickness is less than the second thickness.
9. The clot treatment device of example 1 wherein the proximal mouth is configured to capture a first portion of the clot material, and wherein the third region further defines one or more distal mouths configured to capture a second portion of the clot material.
10. The clot treatment device of example 9 wherein the one or more distal mouths are positioned between the one or more relief features and the distal terminus.
11. The clot treatment device of example 1 wherein the one or more relief features are one or more first relief features, the threshold is a first threshold, and the force is a first force; and wherein the fourth region further comprises a second relief feature configured to (i) retain captured clot material when a second force on the second relief feature is below a second threshold and (ii) release the captured clot material when the second force equals or exceeds the second threshold.
12. The clot treatment device of example 11 wherein the second relief feature includes a portion of the unitary structure that tapers inwardly toward the central axis.
13. The clot treatment device of example 11 wherein the second relief feature includes a collar and a fuse, wherein the collar is coupled to one of the interconnected struts via the fuse to retain the captured clot material proximal to the second relief feature, and wherein, when the second force equals or exceeds the second threshold, the fuse is configured to break to allow the second relief feature to release the capture clot material.
14. The clot treatment device of example 11 wherein the second relief feature includes a pair of struts that terminate at a junction and are angled inwardly toward the central axis.
15. The clot treatment device of example 1 wherein the coupling portion includes a terminal strut coupled to the second region and an attachment strut extending proximally from the terminal strut, wherein the attachment strut defines one or more strut portions that extend at least generally perpendicularly to a length of the catheter to which the coupling portion is configured to be coupled.
16. The clot treatment device of example 15 wherein, in response to one or more forces on the unitary structure that cause the catheter to flex or deform, the attachment strut is configured to flex or deform with the catheter.
17. The clot treatment device of example 15 wherein the attachment strut is configured to extend helically around the catheter.
18. The clot treatment device of example 15 wherein the terminal strut is a distal terminal strut, wherein the coupling portion further includes a proximal terminal strut, and wherein the attachment strut extends between the distal and proximal terminal struts.
19. The clot treatment device of example 15, further comprising an adhesive material applied to the attachment strut to bond the coupling portion to the catheter, wherein, when bonded to the catheter, the attachment strut does not move relative to the catheter.
20 The clot treatment device of example 1 wherein the distal terminus defines a free end of the unitary structure.
21. A system for removing clot material from a blood vessel of patient, the system comprising:
a delivery catheter;
an intermediate catheter configured to extend through the delivery catheter;
an embolic protection device coupled to a distal portion of the intermediate catheter, wherein the delivery catheter and intermediate catheter are movable relative to one another to move the embolic protection device between (a) a first embolic protection device position in which the embolic protection device is constrained within the delivery catheter and (b) a second embolic protection device position in which the embolic protection device is unconstrained by the delivery catheter and configured to expand within the blood vessel;
a deployment catheter configured to extend through the intermediate catheter;
an elongate shaft configured to extend through the deployment catheter; and
a clot treatment device according to any one of examples 1-20, wherein the deployment catheter and the intermediate catheter are movable relative to one another to move the embolic protection device between (a) a first clot treatment device position in which the clot treatment device is constrained within the deployment catheter and (b) a second clot treatment device position in which the clot treatment device is unconstrained by the deployment catheter and configured to expand within the blood vessel.
22. The system of example 21 wherein, in the second embolic protection device position, the embolic protection device is configured to expand to a diameter of the blood vessel.
23. The system of example 22 wherein the embolic protection device is impermeable to blood.
24. The system of example 22 or example 23 wherein, in the second embolic protection device position, the embolic protection device is configured to substantially prevent blood flow through the blood vessel.
25. The system of any one of examples 21-24 wherein the blood vessel is a periphery artery.
26. The system of any one of examples 21-25 wherein the embolic protection device is a funnel having a tapered shape.
27. The system of example 26 wherein the embolic protection device is impermeable to blood.
28. The system of any one of examples 21-27 wherein the embolic protection device is a balloon.
29 The system of example 28 wherein, in the second embolic protection device position, the balloon is configured to expand to a diameter of the blood vessel to substantially prevent blood flow through the blood vessel.
30. The system of any one of examples 21-29, further comprising:
a valve assembly fixedly coupled to a proximal portion of the deployment catheter; and
a hub fixedly coupled to a proximal portion of the elongate shaft, wherein the valve assembly is movable relative to the hub to retract the deployment catheter relative to the elongate shaft to move the clot treatment device between the first clot treatment position and the second clot treatment position.
31. The system of any one of examples 21-30, further comprising a handle having a housing and a trigger movable relative to the housing, wherein the deployment catheter is fixedly coupled to the trigger, and wherein the trigger is movable relative to housing to retract the deployment catheter relative to the elongate shaft to move the clot treatment device between the first clot treatment position and the second clot treatment position.
32. The system of example 31, further comprising a hub fixedly coupled to a proximal portion of the elongate shaft, wherein the hub is coupled to the housing.
33. The system of any one of examples 21-32, further comprising an aspiration source fluidly coupled to the delivery catheter.
34. A method of removing clot material from a blood vessel of a patient, the method comprising:
positioning a deployment catheter within the blood vessel with at least a portion of the deployment catheter distal to the clot material;
retracting the deployment catheter over a shaft relative to a clot treatment device to allow the clot treatment device to expand within the blood vessel at least partially distal to the clot material;
withdrawing the clot treatment device proximally through the clot material to—
   capture a portion of the clot material via a proximal mouth of the clot treatment device, and
   retain the captured portion of the clot material within the clot treatment device and proximal to one or more relief features of the clot treatment device; and
when a force on the one or more relief features equals or exceeds a corresponding threshold, causing the one or more relief features to move from a first orientation to a second orientation to release the captured portion of the clot material.
35 The method of example 34 wherein causing the one or more relief features to move from the first orientation to the second orientation includes causing the one or more relief features to pivot outwardly from an inwardly-angled orientation.
36 The method of example 34 or example 35 wherein the captured portion of the clot material is a first portion of the clot material, and wherein withdrawing the clot treatment device through the clot material further includes—
capturing a second portion of the clot material via a distal mouth of the clot treatment device, the distal mouth positioned distal to the proximal mouth; and
retaining the captured second portion of the clot material within the clot treatment device and proximal to a second relief feature of the clot treatment device.
37. The method of example 36 wherein, when a second force on the second relief feature equals or exceeds a corresponding second threshold, causing the one or more relief features to move from a first orientation to a second orientation to release the capture portion of the clot material.
38 The method of any of examples 34-37 wherein the one or more relief features are one or more first relief features, and wherein the method further comprises, after causing the one or more first relief features to release the portion of the clot material, retaining the portion of the clot material within the clot treatment device and proximal to a second relief feature of the clot treatment device positioned distal to the one or more first relief features.
39 The method of any of examples 34-38 wherein—
the clot treatment device includes a leading edge portion defining the proximal mouth;
the leading edge portion includes a first segment, a second segment distal to the first segment, and a third segment distal to the second segment; and
withdrawing the clot treatment device further includes collapsing the proximal mouth at the third segment, the second segment, and then the first segment in series.
40 The method of any of examples 34-39 wherein causing the one or more relief features to release the captured portion of the clot material includes causing the one or more relief features to release the captured portion of the clot material before one or more other portions of the clot treatment device yields or breaks.
41. The method of any of examples 34-40, further comprising:
before positioning the deployment catheter within the blood vessel—
   advancing a delivery catheter through the blood vessel such that a distal portion of the delivery catheter is proximate to the clot material;
   advancing an embolic protection device through the delivery catheter, wherein the embolic protection device is coupled to a distal portion of an intermediate catheter; and
   retracting the delivery catheter to allow the embolic protection device to expand within the blood vessel at least partially proximal to the clot material;
wherein withdrawing the clot treatment device includes withdrawing the clot treatment device at least partially into the embolic protection device.
42. The method of any of examples 34-41, wherein the one or more relief features include a frangible strut section of the clot treatment device configured to break when the force equals or exceeds the threshold, and wherein causing the one or more relief features to release the captured portion of the clot material includes breaking the frangible strut section.
43 The method of any of examples 34-42, further comprising advancing a cleaning tool through the clot treatment device to mechanically disrupt clot material contained within and/or coupled to the clot treatment device.
44. A method of removing clot material from a blood vessel of a patient, the method comprising:
advancing a delivery catheter through the blood vessel such that a distal portion of the delivery catheter is proximate to the clot material;
advancing an embolic protection device through the delivery catheter, wherein the embolic protection device is coupled to a distal portion of an intermediate catheter;
retracting the delivery catheter to allow the embolic protection device to expand within the blood vessel at least partially proximal to the clot material;
advancing a clot treatment assembly through the intermediate catheter and at least partially through the clot material;
retracting a deployment catheter of the clot treatment assembly relative to a clot treatment device according to any one of examples 1-20 to allow the clot treatment device to expand within the blood vessel at least partially distal to the clot material; and
withdrawing the clot treatment device proximally though the clot material and into the embolic protection device.
45. The method of example 44 wherein the method further comprises substantially inhibiting blood flow through the blood vessel after expanding the embolic protection device.
46. The method of example 44 or example 45 wherein allowing the embolic protection device to expand includes allowing the embolic protection device to expand to a diameter of the blood vessel.
47. The method of any one of examples 44-46 wherein the embolic protection device is impermeable to blood.
48. The system of any one of examples 44-47 wherein the blood vessel is a periphery artery.
49. The system of any one of examples 44-48 wherein the embolic protection device is a funnel having a tapered shape.
50. The system of any one of examples 44-49 wherein the embolic protection device is a balloon, and wherein allowing the embolic protection device to expand includes inflating the balloon.
51. A system for removing clot material from a blood vessel of patient, the system comprising:
a delivery catheter;
an intermediate catheter configured to extend through the delivery catheter;
an embolic protection device coupled to a distal portion of the intermediate catheter, wherein the delivery catheter and intermediate catheter are movable relative to one another to move the embolic protection device between (a) a first embolic protection device position in which the embolic protection device is constrained within the delivery catheter and (b) a second embolic protection device position in which the embolic protection device is unconstrained by the delivery catheter and configured to expand within the blood vessel;
a deployment catheter configured to extend through the intermediate catheter;
an elongate shaft configured to extend through the deployment catheter; and
a clot treatment device according to any one of examples 1-20, wherein the deployment catheter and the intermediate catheter are movable relative to one another to move the embolic protection device between (a) a first clot treatment device position in which the clot treatment device is constrained within the deployment catheter and (b) a second clot treatment device position in which the clot treatment device is unconstrained by the deployment catheter and configured to expand within the blood vessel.
52. The system of example 51 wherein the embolic protection device includes:
a funnel; and
a coating at least partially encapsulating the funnel, wherein the coating is configured to substantially prevent blood flow through the blood vessel and has a thickness that varies along the length of the funnel.
53. The system of example 52 wherein the funnel has a distal end portion and a proximal end portion opposite the first end portion, wherein the coating has a first thickness at the distal end portion and a second thickness greater than the first thickness at the proximal end portion.
54. The system of any of examples 51-53 wherein the embolic protection device includes a plurality of interconnected struts, and wherein a density of the struts varies along the length of the embolic protection device.
55. The system of example 54 wherein—
the embolic protection device has a distal end portion and a proximal end portion opposite the distal end portion,
at the distal end portion, the struts have a first density, and
at the proximal end portion, the struts have a second density less than the first density.
56. The system of any one of examples 51-54 wherein the embolic protection device includes—
one or more first wires configured to extend along and around a longitudinal axis of the intermediate catheter in a first direction;
one or more second wires that extend along and around the longitudinal axis of the intermediate catheter in a second direction opposite the first direction; and
a coupling portion configured to be coupled to an exterior surface portion of the intermediate catheter,
wherein, in the coupling portion, the one or more first wires do not overlap the one or more second wires.
57. The system of example 56 wherein the one or more first wires do not extend into the coupling portion.
58. The system of any of examples 51-57, wherein the embolic protection device further includes a bump feature that, when deployed, defines a maximum radial extent of the embolic protection device.
59. The system of example 58 wherein the bump feature is a widened portion of the embolic protection device.
60. The system of example 58 wherein the embolic protection device further includes a funnel, and wherein the funnel is positioned distal to the bump feature.
61. A balloon catheter, comprising:
an inflation port positioned on a proximal end of the balloon catheter and fluidly connected to a balloon on a distal end thereof; and
an inflation control device fluidly connected to the inflation port, wherein the inflation control device is configured to reduce pressure within the balloon during inflation of the balloon.
62. The balloon catheter of example 61, wherein the inflation control device is selected from a group comprising a pressure relief valve, a flow restrictor valve, and combinations thereof.
63. The balloon catheter of example 62, wherein the pressure relief valve comprises:
an inlet, an outlet, and a flow path fluidly connected therebetween;
a relief outlet defining a relief fluid path that is fluidly connected to the flow path; and
a biasing member and a stopper positioned along the relief fluid path,
wherein the stopper is configured to move between a first position and a second position when a predetermined condition is satisfied. 64 The balloon catheter of example 63, wherein the predetermined condition is a pressure above about 10 psi.
65. The balloon catheter of example 62, wherein the flow restrictor valve comprises:
an inlet, an outlet, and a flow path fluidly connected therebetween; and
a restrictor defining a first flow passage and a second flow passage, the restrictor being positioned along the flow path, wherein the restrictor is configured to move between a first position and a second position,
wherein fluid flow in a first direction travels through the first flow passage of the restrictor and fluid flow in a second direction travels through both the first flow passage and the second flow passage of the restrictor.
66. The balloon catheter of example 62, wherein the flow restrictor valve comprises:
an inlet, an outlet, and a flow path fluidly connected therebetween; and
a flap defining a flow passage, the flap being positioned along the flow path, wherein the flap is configured to move between a first position and a second position,
wherein fluid flow in a first direction travels through the flow passage of the flap and fluid flow in a second direction travels through an opening defined by the flap being in the second position.
67. An inflation control device for a balloon catheter comprising:
an inlet, an outlet and a flow path fluidly connected therebetween;
a relief outlet defining a relief fluid path that is fluidly connected to the flow path; and
a biasing member and a stopper positioned along the relief fluid path,
wherein the stopper is configured to move between a first position and a second position when a predetermined condition is satisfied.
68. An inflation control device for a balloon catheter comprising:
an inlet, an outlet, and a flow path fluidly connected therebetween; and
a restrictor defining a first flow passage positioned along the flow path, wherein the restrictor is configured to move between a first position and a second position,
wherein fluid flow in a first direction travels through the first flow passage of the restrictor.
69. The inflation control device of example 68 wherein the restrictor further comprises a second flow passage positioned along the flow path, wherein fluid flow in a second direction travels through both the first flow passage and the second flow passage.
70. An inflation control device for a balloon catheter comprising:
an inlet, an outlet, and a flow path fluidly connected therebetween; and
a flap defining a flow passage, the flap being positioned along the flow path, wherein the flap is configured to move between a first position and a second position,
wherein fluid flow in a first direction travels through the flow passage of the flap and fluid flow in a second direction travels through an opening defined by the flap being in the second position.
71. The inflation control device of example 70, wherein the flap in the first position is sealed against a seal feature positioned along the flow path.
72. An inflation control device, comprising:
a housing including a flow path extending between an inlet and an outlet; and
a restrictor positioned in the flow path between the inlet and the outlet, wherein the restrictor is configured to move to a first position when fluid flows along the flow path from the inlet to the outlet, wherein the restrictor is configured to move to a second position when the fluid flows along the flow path from the outlet to the inlet, wherein the restrictor defines one or more first fluid channels and a second fluid channel, and wherein—
   in the first position the one or more first fluid channels are blocked such that the fluid flows from the inlet to the outlet through the second fluid channel, and
   in the second position, the one or more first fluid channels are positioned such that the fluid flows from the outlet to the inlet through the one or more first fluid channels and the second fluid channel.
73. The inflation control device of example 72 wherein the one or more first fluid channels comprise slots positioned on an outer surface of the restrictor, and wherein the one or more first fluid channels extend parallel to a longitudinal axis of the inflation control device.
74 The inflation control device of either example 72 or example 73 wherein the second fluid channel comprises a circular lumen extending through the restrictor along a longitudinal axis of the inflation control device.
75. The inflation control device of any of examples 72-74 wherein a flow rate of the fluid through the restrictor is greater in the second position than in the first position.
76 The inflation control device of example 75 wherein the flow rate in the first position is about 0.1 cubic centimeters per second or less.
77. The inflation control device of any of examples 72-76 wherein the inflation control device is configured to be fluidly coupled to an inflatable balloon such that fluid flows more quickly through the inflation control device during deflation of the inflatable balloon than during inflation of the inflatable balloon.
78. The inflation control device of example 77 wherein the inflatable balloon is configured to expand to a diameter of a blood vessel to substantially prevent blood flow through the blood vessel when inflated.
79. A balloon catheter system, comprising:
a balloon catheter including a proximal end and a distal end;
an inflation port positioned at the proximal end of the balloon catheter;
a balloon positioned at the distal end of the balloon catheter and fluidly coupled to the inflation port via the balloon catheter; and
an inflation control device fluidly coupled to the inflation port, the inflation control device including—
   a housing defining an inlet and an outlet in fluid communication with the inlet, and
   a restrictor positioned between the inlet and the outlet, wherein the restrictor is configured to move between a first position and a second position, and wherein—
      in the first position, the restrictor is positioned closer to the outlet than the inlet,
      in the second position, the restrictor is positioned closer to the inlet than the outlet, and
      the restrictor is configured such that fluid flows more quickly through the inflation control device in the second position than in the first position.
80 The balloon catheter system of example 79 wherein the restrictor is movable between the first position and the second position along a longitudinal axis of the inflation control device.
81. The balloon catheter system of either example 79 or example 80 wherein the restrictor is configured to move to the first position when fluid flows from the inlet to the outlet.
82. The balloon catheter system of example 81 wherein the outlet of the inflation control device is directly coupled to the inflation port.
83 The balloon catheter system of any of examples 79-82 wherein the restrictor includes one or more first fluid channels positioned along an outer surface of the restrictor and a second fluid channel extending through the restrictor along a longitudinal axis of the inflation control device.
84 The balloon catheter system of any of examples 79-83, further comprising a second inflation control device fluidly coupled to the inflation port, wherein the second inflation control device is a pressure relief valve configured to divert fluid out of the balloon catheter system when pressure in the balloon catheter exceeds a predetermined pressure.
85. The balloon catheter system of any of examples 79-84, further comprising a deployment catheter extending through the balloon catheter and housing a clot treatment device deployable within a blood vessel.
86. A method comprising:
providing an inflation control device having an inlet, an outlet, and a restrictor positioned between the inlet and the outlet;
connecting a pressurizable fluid source to an inlet of an inflation control device;
connecting a balloon catheter to an outlet of the inflation control device, wherein the balloon catheter is fluidly coupled to a balloon;
injecting fluid from the pressurizable fluid source through the inflation control device by generating positive pressure in the pressurizable fluid source, which causes a restrictor of the inflation control device to move to a first position where fluid flows through the inflation control device at a first flow rate; and
aspirating fluid from the balloon catheter through the inflation control device by generating negative pressure in the pressurizable fluid source, which causes the restrictor to move to a second position where fluid flows through the inflation control device at a second flow rate greater than the first flow rate.
87. The method of example 86 wherein the restrictor defines one or more first fluid channels positioned along an outer surface of the restrictor and a second fluid channel extending through the restrictor along a longitudinal axis of the inflation control device.
88. The method of example 87 wherein moving the restrictor to the first position causes the one or more first fluid channels to be blocked, such that the fluid cannot flow through the one or more first fluid channels.
89 The method of example 87 wherein aspirating the fluid from the balloon catheter through the inflation control device includes aspirating the fluid through the one or more first fluid channels and the second fluid channel.
90. The method of any of examples 86-89 wherein the second flow rate is about 0.1 cubic centimeters per second or less.
91. The method of any of examples 86-90, further comprising:
deploying a clot treatment device from a deployment catheter extending through the balloon catheter.
92. A balloon catheter system, comprising:
a balloon catheter including a proximal end and a distal end;
an inflation port positioned at the proximal end of the balloon catheter;
a balloon positioned at the distal end of the balloon catheter and fluidly coupled to the inflation port via the balloon catheter;
a pressure relief valve fluidly coupled to the inflation portion; and
an inflation control device fluidly coupled to the pressure relief valve, the inflation control device including—
   a housing defining an inlet and an outlet in fluid communication with the inlet, and
   a restrictor positioned between the inlet and the outlet, wherein the restrictor is configured to move between a first position and a second position, and wherein—
      in the first position, the restrictor is positioned closer to the outlet than the inlet,
      in the second position, the restrictor is positioned closer to the inlet than the outlet, and
      the restrictor is configured such that fluid flows more quickly through the inflation control device in the second position than in the first position.
93. A balloon catheter system, comprising:
a balloon catheter including a proximal end and a distal end;
an inflation port positioned at the proximal end of the balloon catheter;
a balloon positioned at the distal end of the balloon catheter and fluidly coupled to the inflation port via the balloon catheter; and
a pressure relief valve fluidly coupled to the inflation port, the pressure relief valve including—
   a housing defining an inlet, an outlet in fluid communication with the inlet and fluidly coupled to the inflation port, and a relief outlet positioned between and fluidly coupled to the inlet and the outlet, wherein the relief outlet is configured to move between a first position and a second position, and wherein—
      in the first position, the relief outlet is closed such that fluid cannot flow into the relief outlet, and
      in the second position, the relief outlet is open such that fluid can flow into the relief outlet and out of the pressure relief valve.
94. The balloon catheter system of example 93, the pressure relief valve further comprising:
a stopper positioned in an opening of the relief outlet and biased towards the opening.
95 The balloon catheter system of example 94 wherein a biasing force of a biasing member biasing the stopper toward the opening is equal to a desired cracking pressure of the balloon catheter system.
96. The balloon catheter system of any of examples 93-95 wherein the relief outlet is fluidly coupled to a collection reservoir spaced apart from the inflation port.
97. The balloon catheter system of any of examples 93-96, further comprising:
an inflation control device fluidly coupled to the inlet of the pressure relief valve, the inflation control device including—
   a second housing defining a second inlet and a second outlet in fluid communication with the second inlet, and
   a restrictor positioned between the second inlet and the second outlet, wherein the restrictor is configured to move between a first position and a second position, and wherein—
      in the first position, the restrictor is positioned closer to the second outlet than the second inlet,
      in the second position, the restrictor is positioned closer to the second inlet than the second outlet, and
      the restrictor is configured such that fluid flows more quickly through the inflation control device in the second position than in the first position.
98. The balloon catheter system of any of examples 93-97, further comprising a deployment catheter extending through the balloon catheter and housing a clot treatment device.

### V. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An inflation control device, comprising:
a housing including a flow path extending between an inlet and an outlet; and
a restrictor positioned in the flow path between the inlet and the outlet, wherein the restrictor is configured to move to a first position when fluid flows along the flow path from the inlet to the outlet, wherein the restrictor is configured to move to a second position when the fluid flows along the flow path from the outlet to the inlet, wherein the restrictor defines one or more first fluid channels and a second fluid channel, and wherein—
in the first position the one or more first fluid channels are blocked such that the fluid flows from the inlet to the outlet through the second fluid channel, and
in the second position, the one or more first fluid channels are positioned such that the fluid flows from the outlet to the inlet through the one or more first fluid channels and the second fluid channel.

2. The inflation control device of claim 1 wherein the one or more first fluid channels comprise slots positioned on an outer surface of the restrictor, and wherein the one or more first fluid channels extend parallel to a longitudinal axis of the inflation control device.

3. The inflation control device of claim 1 wherein the second fluid channel comprises a circular lumen extending through the restrictor along a longitudinal axis of the inflation control device.

4. The inflation control device of claim 1 wherein a flow rate of the fluid through the restrictor is greater in the second position than in the first position.

5. The inflation control device of claim 4 wherein the flow rate in the first position is about 0.1 cubic centimeters per second or less.

6. The inflation control device of claim 1 wherein the inflation control device is configured to be fluidly coupled to an inflatable balloon such that fluid flows more quickly through the inflation control device during deflation of the inflatable balloon than during inflation of the inflatable balloon.

7. The inflation control device of claim 6 wherein the inflatable balloon is configured to expand to a diameter of a blood vessel to substantially prevent blood flow through the blood vessel when inflated.

8. A balloon catheter system, comprising:
a balloon catheter including a proximal end and a distal end;
an inflation port positioned at the proximal end of the balloon catheter;
a balloon positioned at the distal end of the balloon catheter and fluidly coupled to the inflation port via the balloon catheter; and
an inflation control device fluidly coupled to the inflation port, the inflation control device including—
a housing defining an inlet and an outlet in fluid communication with the inlet, and
a restrictor positioned between the inlet and the outlet, wherein the restrictor is configured to move between a first position and a second position, and wherein—
in the first position, the restrictor is positioned closer to the outlet than the inlet,
in the second position, the restrictor is positioned closer to the inlet than the outlet, and
the restrictor is configured such that fluid flows more quickly through the inflation control device in the second position than in the first position.

9. The balloon catheter system of claim 8 wherein the restrictor is movable between the first position and the second position along a longitudinal axis of the inflation control device.

10. The balloon catheter system of claim 8 wherein the restrictor is configured to move to the first position when fluid flows from the inlet to the outlet.

11. The balloon catheter system of claim 10 wherein the outlet of the inflation control device is directly coupled to the inflation port.

12. The balloon catheter system of claim 8 wherein the restrictor includes one or more first fluid channels positioned along an outer surface of the restrictor and a second fluid channel extending through the restrictor along a longitudinal axis of the inflation control device.

13. The balloon catheter system of claim 8, further comprising a second inflation control device fluidly coupled to the inflation port, wherein the second inflation control device is a pressure relief valve configured to divert fluid out of the balloon catheter system when pressure in the balloon catheter exceeds a predetermined pressure.

14. The balloon catheter system of claim 8, further comprising a deployment catheter extending through the balloon catheter and housing a clot treatment device deployable within a blood vessel.

15. A balloon catheter system, comprising:
a balloon catheter including a proximal end and a distal end;
an inflation port positioned at the proximal end of the balloon catheter;
a balloon positioned at the distal end of the balloon catheter and fluidly coupled to the inflation port via the balloon catheter; and
a pressure relief valve fluidly coupled to the inflation port, the pressure relief valve including—
a housing defining an inlet, an outlet in fluid communication with the inlet and fluidly coupled to the inflation port, and a relief outlet positioned between and fluidly coupled to the inlet and the outlet, wherein the relief outlet is configured to move between a first position and a second position, and wherein—
in the first position, the relief outlet is closed such that fluid cannot flow into the relief outlet, and
in the second position, the relief outlet is open such that fluid can flow into the relief outlet and out of the pressure relief valve.

16. The balloon catheter system of claim 15, the pressure relief valve further comprising:
a stopper positioned in an opening of the relief outlet and biased towards the opening.

17. The balloon catheter system of claim 16 wherein a biasing force of a biasing member biasing the stopper toward the opening is equal to a desired cracking pressure of the balloon catheter system.

18. The balloon catheter system of claim 15 wherein the relief outlet is fluidly coupled to a collection reservoir spaced apart from the inflation port.

19. The balloon catheter system of claim 15, further comprising:
an inflation control device fluidly coupled to the inlet of the pressure relief valve, the inflation control device including—
a second housing defining a second inlet and a second outlet in fluid communication with the second inlet, and
a restrictor positioned between the second inlet and the second outlet, wherein the restrictor is configured to move between a first position and a second position, and wherein—
in the first position, the restrictor is positioned closer to the second outlet than the second inlet,
in the second position, the restrictor is positioned closer to the second inlet than the second outlet, and
the restrictor is configured such that fluid flows more quickly through the inflation control device in the second position than in the first position.

20. The balloon catheter system of claim 15, further comprising a deployment catheter extending through the balloon catheter and housing a clot treatment device.
